(19) United States
(12) Patent Application Publication
(10) Pub. No.: US 2009/0047211 A1
(43) Pub. Date: Feb. 19, 2009

(54) Anti-mesothelin antibodies useful for immunological assays

(75) Inventors: Ira H. Pastan, Potomac, MD (US); Masanori Onda, Rockville, MD (US)

Correspondence Address: TOWNSEND AND TOWNSEND AND CREW, LLP, TWO EMBARCADERO CENTER, 8TH FLOOR, SAN FRANCISCO, CA, 94111, US

(73) Assignee: The Govt. of the U.S. as represented by the Sec. of the Dept. of Health and Human Services, Rockville, MD (US)

(21) Appl. No.: 11/920,222

(22) PCT Filed: May 11, 2006
(86) PCT No.: PCT/US2006/018502

§ 371 Date: Nov. 9, 2007

Related U.S. Application Data
(60) Provisional application No. 60/681,104, filed on May 12, 2005.

(51) Int. Cl. A61K 51/00; C07K 16/18; A61K 9/127; C12N 15/00; G01N 33/53; A61P 31/00; C12N 5/06; C12N 15/11; A61K 39/395

(52) U.S. Cl. 424/1.49; 530/387.3; 424/178.1; 530/391.3; 530/391.7; 424/450; 536/23.53; 435/320.1; 435/375; 435/7.2

(57) ABSTRACT

The present invention provides antibodies that have a surprisingly good combination of affinity for mesothelin and ability to be used in immunological assays for detecting the presence of mesothelin in biological samples. The invention further provides methods of using the antibodies, and kits comprising them. The antibodies can also be used to target toxins and other agents to cells expressing mesothelin, and can be used in methods and medicaments for inhibiting the growth of such cells.

### CROSS-REFERENCES TO RELATED APPLICATIONS

This application claims the benefit of U.S. Provisional Application No. 60/681,104, filed May 12, 2005, the contents of which are incorporated herein by reference.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

Mesothelin is a 40 kDa glycosylphosphatidylinositol linked cell surface glycoprotein present on normal mesothelial cells that is highly expressed in mesothelioma, ovarian cancer, pancreatic cancer, and some other malignancies (Chang, K. et al., Am J Surg Pathol 16:259-68 (1992); Chang, K. et al., Int J Cancer 50:373-81 (1992); Argani, P. et al., Clin Cancer Res 7:3862-8 (2001); Chang, K. et al., Proc Natl Acad Sci USA 93:136-40 (1996)). The normal biological function of mesothelin is unknown and mesothelin deficient mice have no phenotype (Bera, T. K. et al., Mol Cell Biol 20:2902-6 (2000)). However, a recent report indicates that mesothelin can bind to CA125/MUC16, suggesting that mesothelin might have a role in the metastatic spread of ovarian cancer (Rump, A. et al., J Biol Chem 279:9190-8 (2004)).

Initial studies of mesothelin expression were performed by immunohistochemistry with monoclonal antibody (MAb) K1 generated by immunization of mice with the human ovarian carcinoma cell line OVCAR-3. These studies demonstrated that mesothelin is highly expressed in many human cancers including ovarian cancers, mesotheliomas, and squamous cell cancers. The studies were performed on frozen sections, because MAb K1 did not work well on fixed tissues. However, using antigen retrieval by incubating tissue sections in 3M urea, mesothelin expression can be detected in paraffin embedded formalin fixed tissues.

Subsequently, MAb 5B2 was generated by immunizing mice with a recombinant prokaryotic fusion protein corresponding to 100 amino acids which are present in the amino terminus of mesothelin (Hassan, R. et al., Clin Cancer Res 10:3937-42 (2004)). Several immunohistochemical studies have been performed with MAb 5B2 (Ordonez, N. G. Mol Pathol 16:192-7 (2003); Ordonez, N. G. Am J Surg Pathol 27:1418-28 (2003)). The studies confirmed that mesothelin is expressed in mesotheliomas, ovarian cancers and squamous cell tumors and also showed expression in pancreatic cancer and lung cancer, although the distribution in lung cancer was diffuse and not limited to the plasma membrane. No expression of mesothelin was observed in lung cancer using MAb K1 (Chang, K. et al., Am J Surg Pathol 16:259-68 (1992)).

Because of its high expression in cancers and limited expression on normal tissues, mesothelin is also a promising target for cancer immunotherapy. Immunotoxins are chimeric proteins composed of the Fv portion of an antibody fused to a 38 kDa fragment of Pseudomonas exotoxin A (this truncated form is referred to as “PE38”). One immunotoxin, BL22, targets the cell surface protein CD22, and has produced many complete remissions in drug resistant Hairy Cell Leukemia, showing these agents can be useful in treating cancers in humans (Kreitman, R. J. et al., N Engl J Med 345:241-7 (2001)). SS1 (dsFv)-PE38 (also known as “SS1P”) is an immunotoxin composed of (i) an antibody fragment reacting with mesothelin and (ii) PE38, for the treatment of mesothelin expressing cancers. SS 1P has been shown to specifically kill mesothelin expressing cell lines and to cause regressions of mesothelin expressing tumors in mice (Hassan, R. et al., Clin Cancer Res 8:3520-6 (2002); Onda, M. et al., Cancer Res 61:5070-7 (2001)). Based on these studies and appropriate safety data, 2 phase I trials with SS1P are being conducted at the National Cancer Institute in patients with mesothelin expressing cancers (Chowdhury, P. S. et al., Proc Natl Acad Sci USA 95:669-74 (1998); Hassan, R. et al., Proc Am Soc Clin Oncol 21:29a (2002)). In addition, other therapies targeting mesothelin are in preclinical development (Thomas, A. M. et al., J Exp Med 200:297-306 (2004)).

Hellstrom and colleagues developed an ELISA based assay to detect a protein in the blood they term “soluble mesothelin related protein” (or “SMR”) (Scholler, N. et al., Proc Natl Acad Sci USA 96:11531-6 (1999)). Interestingly, SMR is elevated in many patients with mesothelioma and some patients with ovarian cancer. The level of SMR in the blood has been found to fall after effective treatment in mesothelioma (Robinson, B. W. S. et al., Lancet 362:1612-6 (2003)). The identity of the SMR protein has not been established, but is not considered to be mesothelin.

Research in the mesothelin area has been hampered by the lack of well-characterized, readily available antibodies that could be used for immunohistochemistry on fixed tissues, Western blotting, FACS analysis of cells from patients and ELISA to measure mesothelin in the blood and body fluids and other purposes. The present invention fills these and other needs.

### BRIEF SUMMARY OF THE INVENTION

In a first group of embodiments, the invention provides isolated antibodies comprising a variable heavy (VH) chain and a variable light (VL) chain, which VH and VL chains have 90% or greater identity to SEQ ID NOS:1 and 3, respectively, or to the VH and VL chains of antibody MB, ATCC Patent Deposit Designation PTA-6709, respectively, and which specifically bind mesothelin. In some embodiments, the VH and VL chains have identity to SEQ ID NOS: 1 and 3, respectively, or to the VH and VL chains of antibody MB, ATCC Patent Deposit Designation PTA-6709, of 95% or greater. In some embodiments, the VH and said VL chains each have complementarity determining regions (CDRs) 1, 2, and 3, wherein (a) CDRs 1, 2, and 3 of the VH chain have the sequences shown in FIG. 1 with respect to SEQ ID NO:1 and which CDRs 1, 2, and 3 of the VL chain have the sequences shown in FIG. 1, with respect to SEQ ID NO:3, or (b) CDRs 1, 2, and 3 of said VH chain have the sequences of the corresponding CDRs of antibody MB, ATCC Patent Deposit Designation PTA-6709, and CDRs 1, 2, and 3 of the VL chain have the sequences of the corresponding CDRs of antibody MB, ATCC Patent Deposit Designation PTA-6709. In some embodiments, the antibody comprises SEQ ID NOS:1 and 3 joined by a peptide linker or the VH and VL chains of antibody MB, ATCC Patent Deposit Designation PTA-6709, joined by a peptide linker. In some embodiments, the VH and said VL chains are connected by a disulfide bond between a cysteine residue engineered into each chain. In some embodiments, the antibody is selected from the group consisting of an scFv, a dsFv, a diabody, a domain antibody, a Fab, a F(ab′)2 or an intact immunoglobulin. In some embodiments, the VH and the VL chains each have complementarity determining regions (“CDRs”) 1, 2, and 3, wherein CDRs 1, 2, and 3 of the VH chain and CDRs 1, 2, and 3 of the VL chain have the sequences shown in FIG. 1 for antibody MN or the sequence of the corresponding CDRs of antibody MB, ATCC Patent Deposit Designation PTA-6709, respectively, except that (a) one or more CDRs have a mutation of a residue encoded by a codon with a nucleotide falling within (i) a tetranucleotide motif A/G-G-C/T-A/T or (ii) AGY, where Y can be a C or a T, or (b) one or more CDRs have a mutation of a residue that is not encoded by a codon with a nucleotide falling within (i) a tetranucleotide motif A/G-G-C/T-A/T or (ii) AGY, where Y can be a C or a T, or (c) one or more CDRs have a mutation of a residue that is encoded by a codon with a nucleotide falling within (i) a tetranucleotide motif A/G-G-C/T-A/T or (ii) AGY, where Y can be a C or a T, and one or more CDRs have a mutation of a residue that is not encoded by a codon with a nucleotide falling within (i) a tetranucleotide motif A/G-G-C/T-A/T or (ii) AGY, where Y can be a C or a T.

In a further group of embodiments, the invention provides chimeric molecules comprising (a) an isolated antibody which specifically binds mesothelin, which antibody comprises a variable heavy (VH) chain and a variable light (VL) chain, which VH and VL chains have 90% or greater identity to (i) SEQ ID NOS:1 and 3, respectively, or (ii) to the VH and VL chains of antibody MB, ATCC Patent Deposit Designation PTA-6709, respectively, and (b) a therapeutic moiety or a detectable label. In some embodiments, the VH and VL chains have 95% or greater identity to (i) SEQ ID NOS:1 and 3, respectively, or (ii) to the VH and VL chains of antibody MB, ATCC Patent Deposit Designation PTA-6709, respectively. In some embodiments, the VH and VL chains have the sequence of (i) SEQ ID NOS:1 and 3, respectively, or (ii) the VH and VL chains of antibody MB, ATCC Patent Deposit Designation PTA-6709, respectively. In some embodiments, the therapeutic moiety is selected from the group consisting of a cytotoxin, a drug, a radioisotope, or a liposome loaded with a drug or a cytotoxin. n some embodiments, the therapeutic moiety is a cytotoxin is selected from the group consisting of ricin A, abrin, ribotoxin, ribonuclease, saporin, calicheamycin, diphtheria toxin, a Pseudomonas exotoxin (“PE”), and botulinum toxins A through F. In some embodiments, the VH and VL chains each have complementarity determining regions (“CDRs”) 1, 2, and 3, wherein CDRs 1, 2, and 3 of the VH chain and CDRs 1, 2, and 3 of said VL chain have the sequences shown in FIG. 1 for antibody MN or the CDRs of antibody MB, ATCC Patent Deposit Designation PTA-6709, respectively, except that (i) one or more CDRs have a mutation of a residue encoded by a codon with a nucleotide falling within (A) a tetranucleotide motif A/G-G-C/T-A/T or (B) AGY, where Y can be a C or a T, or (ii) one or more CDRs have a mutation of a residue that is not encoded by a codon with a nucleotide falling within (A) a tetranucleotide motif A/G-G-C/T-A/T or (B) AGY, where Y can be a C or a T, or (iii) one or more CDRs have a mutation of a residue that is encoded by a codon with a nucleotide falling within (A) a tetranucleotide motif A/G-G-C/T-A/T or (B) AGY, where Y can be a C or a T, and one or more CDRs have a mutation of a residue that is not encoded by a codon with a nucleotide falling within (C) a tetranucleotide motif A/G-G-C/T-A/T or (D) AGY, where Y can be a C or a T.

In a further group of embodiments, the invention provides compositions comprising any of the chimeric molecules described above, and a pharmaceutically acceptable carrier. In some embodiments, wherein the chimeric molecule includes a therapeutic moiety, the therapeutic moiety is selected from the group consisting of a cytotoxin, a drug, a radioisotope, or a liposome loaded with a drug or a cytotoxin.

In yet a further group of embodiments, the invention provides isolated nucleic acids. The isolated nucleic acid encode an antibody comprising a variable heavy (VH) chain and a variable light (VL) chain, which VH and VL chains have 90% or greater identity to SEQ ID NOS:1 and 3, respectively, or to the VH and VL chains of antibody MB, ATCC Patent Deposit Designation PTA-6709, respectively, and which specifically binds mesothelin. In some embodiments, the identity to SEQ ID NOS:1 and 3, respectively, or to the VH and VL chains of antibody MB, ATCC Patent Deposit Designation PTA-6709, respectively, is 95% or greater. In some embodiments, the VH and VL chains have the sequence of SEQ ID NOS:1 and 3, respectively, or of the VH and VL chains, respectively, of antibody MB, ATCC Patent Deposit Designation PTA-6709. In some embodiments, the VH and said VL chains each have complementarity determining regions (CDRs) 1, 2, and 3, wherein (a) CDRs 1, 2, and 3 of said VH chain have the sequences shown in FIG. 1 with respect to SEQ ID NO:1 and which CDRs 1, 2, and 3 of said VL chain have the sequences shown in FIG. 1, with respect to SEQ ID NO:3, or (b) CDRs 1, 2, and 3 of said VH chain have the sequences of CDRs 1, 2, and 3, respectively, of the VH chain of antibody MB, ATCC Patent Deposit Designation PTA-6709 and which CDRs 1, 2, and 3 of said VL chain have the sequences of CDRs 1, 2, and 3, respectively, of the VL chain of antibody MB, ATCC Patent Deposit Designation PTA-6709. In some embodiments, the VH and said VL chains each have complementarity determining regions (CDRs) 1, 2, and 3, wherein CDRs 1, 2, and 3 of said VH chain and CDRs 1, 2, and 3 of said VL chain have the sequences shown in FIG. 1 for antibody MN, or of antibody MB, ATCC Patent Deposit Designation PTA-6709, respectively, except that (i) one or more CDRs have a mutation of a residue encoded by a codon with a nucleotide falling within (A) a tetranucleotide motif A/G-G-C/T-A/T or (B) AGY, where Y can be a C or a T, or (ii) one or more CDRs have a mutation of a residue that is not encoded by a codon with a nucleotide falling within (A) a tetranucleotide motif A/G-G-C/T-A/T or (B) AGY, where Y can be a C or a T, or (iii) one or more CDRs have a mutation of a residue that is encoded by a codon with a nucleotide falling within (A) a tetranucleotide motif A/G-G-C/T-A/T or (B) AGY, where Y can be a C or a T, and one or more CDRs have a mutation of a residue that is not encoded by a codon with a nucleotide falling within (C) a tetranucleotide motif A/G-G-C/T-A/T or (D) AGY, where Y can be a C or a T. In some embodiments, the antibody encoded by said nucleic acid is selected from the group consisting of an scFv, a dsFv, a Fab, a F(ab′)2, a diabody, a domain antibody, or an intact immunoglobulin. In some embodiments, the nucleic acid further encodes a therapeutic moiety or a detectable label. In some embodiments, the therapeutic moiety is a drug or a cytotoxin. In some embodiments, the cytotoxin is selected from the group consisting of ricin A, abrin, ribotoxin, ribonuclease, saporin, calicheamycin, diphtheria toxin, a Pseudomonas exotoxin (“PE”), and botulinum toxins A through F.

In some embodiments, the invention provides any of the nucleic acids described above, operably linked to a promoter.

In yet a further group of embodiments, the invention provides methods of inhibiting growth of a cell expressing mesothelin, which methods comprise contacting the cell with a chimeric molecule comprising (a) an antibody that binds to mesothelin, which antibody has variable heavy (VH) and variable light (VL), which VH and VL chains have 90% or greater identity to SEQ ID NOS:1 and 3, respectively, or to the VH and VL chains of antibody MB, ATCC Patent Deposit Designation PTA-6709, respectively, and (b) a therapeutic moiety, whereby contacting said cell with said therapeutic moiety inhibits growth of said cell. In some embodiments, the identity to SEQ ID NOS:1 and 3 or to the VH and VL chains, respectively, of antibody MB, ATCC Patent Deposit Designation PTA-6709, is 95% or greater. In some embodiments, the identity to SEQ ID NOS:1 and 3 or to the VH and VL chains, respectively, of antibody MB, ATCC Patent Deposit Designation PTA-6709, is 95% or greater. In some embodiments, the VH and VL chains have the sequence of SEQ ID NOS:1 and 3, respectively, or of the VH and VL chains, respectively, of antibody MB, ATCC Patent Deposit Designation PTA-6709. In some embodiments, the VH and said VL chains each have complementarity determining regions (CDRs) 1, 2, and 3, wherein (a) CDRs 1, 2, and 3 of the V_{H} chain have the sequences shown in FIG. 1 with respect to SEQ ID NO:1 and which CDRs 1, 2, and 3 of said VL chain have the sequences shown in FIG. 1, with respect to SEQ ID NO:3, or (b) CDRs 1, 2, and 3 of said VH chain have the sequences of the CDRs of the VH chain of antibody MB, ATCC Patent Deposit Designation PTA-6709, and CDRs 1, 2, and 3 of said VL chain have the sequences of the CDRs of the VL chain of antibody MB, ATCC Patent Deposit Designation PTA-6709. In some embodiments, the VH and said VL chains each have complementarity determining regions (CDRs) 1, 2, and 3, wherein CDRs 1, 2, and 3 of said VH chain and CDRs 1, 2, and 3 of said VL chain have the sequences shown in FIG. 1 for antibody MN, or of the CDRs of the respective chain of antibody MB, ATCC Patent Deposit Designation PTA-6709, respectively, except that (i) one or more CDRs have a mutation of a residue encoded by a codon with a nucleotide falling within (A) a tetranucleotide motif A/G-G-C/T-A/T or (B) AGY, where Y can be a C or a T, or (ii) one or more CDRs have a mutation of a residue that is not encoded by a codon with a nucleotide falling within (A) a tetranucleotide motif A/G-G-C/T-A/T or (B) AGY, where Y can be a C or a T, or (iii) one or more CDRs have a mutation of a residue that is encoded by a codon with a nucleotide falling within (A) a tetranucleotide motif A/G-G-C/T-A/T or (B) AGY, where Y can be a C or a T, and one or more CDRs have a mutation of a residue that is not encoded by a codon with a nucleotide falling within (C) a tetranucleotide motif A/G-G-C/T-A/T or (D) AGY, where Y can be a C or a T. In some embodiments, the antibody is selected from the group consisting of an scFv, a dsFv, a Fab, a F(ab′)2, a diabody, a domain antibody, or an intact immunoglobulin. In some embodiments, the therapeutic moiety is selected from the group consisting of a cytotoxin, a drug, a radioisotope, or a liposome loaded with a drug or a cytotoxin. In some embodiments, the cytotoxin is selected from the group consisting of ricin A, abrin, ribotoxin, ribonuclease, saporin, calicheamycin, diphtheria toxin, a Pseudomonas exotoxin (“PE”), and botulinum toxins A through F.

In still another group of embodiments, the invention provides methods for detecting the presence of a cell expressing mesothelin in a biological sample. The methods comprise (a) contacting cells of said biological sample with a chimeric molecule comprising (i) an antibody that specifically binds to mesothelin, said antibody having a variable heavy (VH) chain and a variable light (VL) chain, which VH and VL chains have 90% or greater identity to SEQ ID NOS:1 and 3, respectively, or to the VH chain and the VL chain, respectively, of antibody MB, ATCC Patent Deposit Designation PTA-6709 SEQ ID NOS:1 and 3, and (ii) a detectable label; and, (b) detecting the presence or absence of said label, wherein detecting the presence of said label indicates the presence of a mesothelin-expressing cell in the sample. In some embodiments, the identity to SEQ ID NOS:1 and 3, respectively, or to the VH chain and of the VL chain respectively, of antibody MB, ATCC Patent Deposit Designation PTA-6709, is 95% or greater. In some embodiments, the VH chain and the VL chain have the sequence of (i) SEQ ID NOS:1 and 3, respectively, or (ii) or of the VH chain and of the VL chain, respectively, of antibody MB, ATCC Patent Deposit Designation PTA-6709. In some embodiments, the VH and the VL chains each have complementarity determining regions (CDRs) 1, 2, and 3, wherein (a) CDRs 1, 2, and 3 of the VH chain have the sequences shown in FIG. 1 with respect to SEQ ID NO:1 and which CDRs 1, 2, and 3 of said VL chain have the sequences shown in FIG. 1, with respect to SEQ ID NO:3, or (b) CDRs 1, 2, and 3 of said VH chain have the sequences of the corresponding CDRs of the VH chain of antibody MB, ATCC Patent Deposit Designation PTA-6709 and which CDRs 1, 2, and 3 of said VL chain have the sequences of the corresponding CDRs of antibody MB, ATCC Patent Deposit Designation PTA-6709. In some embodiments, the VH and the VL chains each have complementarity determining regions (CDRs) 1, 2, and 3, wherein CDRs 1, 2, and 3 of said VH chain and CDRs 1, 2, and 3 of said VL chain have the sequences shown in FIG. 1 for antibody MN, respectively, or the sequences of the corresponding CDRs of antibody MB, ATCC Patent Deposit Designation PTA-6709, respectively, except that (i) one or more CDRs have a mutation of a residue encoded by a codon with a nucleotide falling within (A) a tetranucleotide motif A/G-G-C/T-A/T or (B) AGY, where Y can be a C or a T, or (ii) one or more CDRs have a mutation of a residue that is not encoded by a codon with a nucleotide falling within (A) a tetranucleotide motif A/G-G-C/T-A/T or (B) AGY, where Y can be a C or a T, or (iii) one or more CDRs have a mutation of a residue that is encoded by a codon with a nucleotide falling within (A) a tetranucleotide motif A/G-G-C/T-A/T or (B) AGY, where Y can be a C or a T, and one or more CDRs have a mutation of a residue that is not encoded by a codon with a nucleotide falling within (C) a tetranucleotide motif A/G-G-C/T-A/T or (D) AGY, where Y can be a C or a T. In some embodiments, the antibody is selected from the group consisting of an scFv, a dsFv, a Fab, a F(ab′)2, a diabody, a domain antibody, or an intact immunoglobulin.

In another group of embodiments, the invention provides kits for detecting the presence of a mesothelin-expressing cell in a biological sample. The kits comprise (a) a container, and (b) a chimeric molecule comprising (i) an antibody that specifically binds to mesothelin, said antibody having a variable heavy (VH) chain and a variable light (VL) chain, which VH and VL chains have 90% or greater identity to SEQ ID NOS:1 and 3, respectively or to the sequences of the VH and the VL chains, respectively, of antibody MB, ATCC Patent Deposit Designation PTA-6709. In some embodiments, the identity to SEQ ID NOS:1 and 3 or to the sequences of the VH and the VL chains, respectively, of antibody MB, ATCC Patent Deposit Designation PTA-6709, is 95% or greater. In some embodiments, the antibody VH and VL chains have the sequence of SEQ ID NOS:1 and 3, respectively or the sequences of the VH and the VL chains, respectively, of antibody MB, ATCC Patent Deposit Designation PTA-6709. In some embodiments, the VH chain and the VL chain each have complementarity determining regions (CDRs) 1, 2, and 3, wherein (a) CDRs 1, 2, and 3 of said VH chain have the sequences shown in FIG. 1 with respect to SEQ ID NO:1 and which CDRs 1, 2, and 3 of said VL chain have the sequences shown in FIG. 1, with respect to SEQ ID NO:3, or (b) CDRs 1, 2, and 3 of said VH chain and which CDRs 1, 2, and 3 of said VL chain have the sequences of the corresponding chain of antibody MB, ATCC Patent Deposit Designation PTA-6709. In some embodiments, the VH and the VL chains each have complementarity determining regions (CDRs) 1, 2, and 3, wherein CDRs 1, 2, and 3 of the VH chain and CDRs 1, 2, and 3 of the VL chain have the sequences shown in FIG. 1 for antibody MN or CDRs 1, 2, and 3 of the corresponding chain of antibody MB, ATCC Patent Deposit Designation PTA-6709, for antibody MB, respectively, except that (i) one or more CDRs have a mutation of a residue encoded by a codon with a nucleotide falling within (A) a tetranucleotide motif A/G-G-C/T-A/T or (B) AGY, where Y can be a C or a T, or (ii) one or more CDRs have a mutation of a residue that is not encoded by a codon with a nucleotide falling within (A) a tetranucleotide motif A/G-G-C/T-A/T or (B) AGY, where Y can be a C or a T, or (iii) one or more CDRs have a mutation of a residue that is encoded by a codon with a nucleotide falling within (A) a tetranucleotide motif A/G-G-C/T-A/T or (B) AGY, where Y can be a C or a T, and one or more CDRs have a mutation of a residue that is not encoded by a codon with a nucleotide falling within (C) a tetranucleotide motif A/G-G-C/T-A/T or (D) AGY, where Y can be a C or a T. In some embodiments, the kit further comprising a detectable label.

### BRIEF DESCRIPTION OF THE DRAWINGS

FIGS. 1A and B. FIG. 1A shows the deduced amino acid sequences of the V_{H} chain of antibody MN (SEQ ID NO:1) and of antibody MB (SEQ ID NO:2). FIG. 1B shows the deduced amino acid sequences of the V_{L} chain of antibody MN (SEQ ID NO:3) and of antibody MB (SEQ ID NO:4).

FIG. 2. FIG. 2 shows the nucleotide sequence of the V_{H} chain (SEQ ID NO:5) of antibody MN and the V_{L} chain (SEQ ID NO:6) of antibody MB, as well as the nucleotide sequence of the V_{H} chain (SEQ ID NO:7) of antibody MN and the sequence of the V_{L} chain (SEQ ID NO:8) of antibody MB.

FIG. 3. FACS analysis of anti-mesothelin MAbs with different cells. Cells were incubated with anti-mesothelin MAbs (1 μg/ml) or with the anti-CD30 MAb, T6, as a negative control, followed by PE-labeled goat anti-mouse IgG. Each histogram shows the staining with first antibody (line) and without first antibody (gray shadow). The first antibody used in each study is indicated on the horizontal axis, while the cell type on which the antibody was tested is indicated on the vertical axis. Mean fluorescence intensities for Table 1 were measured under the same condition for all experiments.

FIGS. 4A and B. MAb reactivity on ELISA plates coated with mesothelin-Fc or bacterial MBP-mesothelin. FIG. 4A shows the results of ELISA plates coated with mesothelin-Fc (2 μg/ml), while FIG. 4B shows the results for plates coated with bacterial mesothelin (2 μg/ml). Both Figures: ELISA plates were then incubated with anti-mesothelin MAbs, MN(O), MB ( [symbol] ), K1 (♦), 5B2 (→) or anti-CD30 MAb, T6 (x), followed by HRP-conjugated goat anti-mouse IgG. Experiments were done in triplicate. Data are expressed as the mean +SD (n=3).

FIG. 5. Western blot analysis of anti-mesothelin MAbs. Mesothelin-Fc (Lane 1, 100 ng; Lane 2, 25 ng; Lane 3, 6 ng; Lane 4, 2 ng; and Lane 5, 0.4 ng) and CD25-Fc (Lane 6, 50 ng) were separated by SDS-PAGE and transferred to PVDF membranes. Membranes were probed with each anti-mesothelin MAbs (1 μg/ml) or anti-CD30 MAb, T6 (control), followed by ALP-goat anti-mouse IgG and BCIP/NBT substrate. Each experiment was done under the same conditions. Data are summarized in Table 1.

FIG. 6. Immunohistochemistry of anti-mesothelin MAbs. Peroxidase immunohistochemical results are shown from the same area of the same case of mesothelioma. Formaldehyde-fixed, paraffin-embedded tissue sections were treated with a standard antigen retrieval procedure (Vector antigen unmasking solution) in panels A, B, C and D, but one section was treated with the 3M urea antigen retrieval as shown in panel A′. Anti-mesothelin primary antibodies were used to label these sections as follows: (A, A′) MAb K1; (B) MAb 5B2; (C) MAb MB; and (D) MAb MN. Note that MAb K1 labels poorly with standard antigen retrieval, but is much stronger using the 3M urea treatment, whereas the other antibodies label well using standard antigen retrieval, with MAb MB (panel C) showing the most intense reaction. (Magnifications=×200; Bar=50 μm).

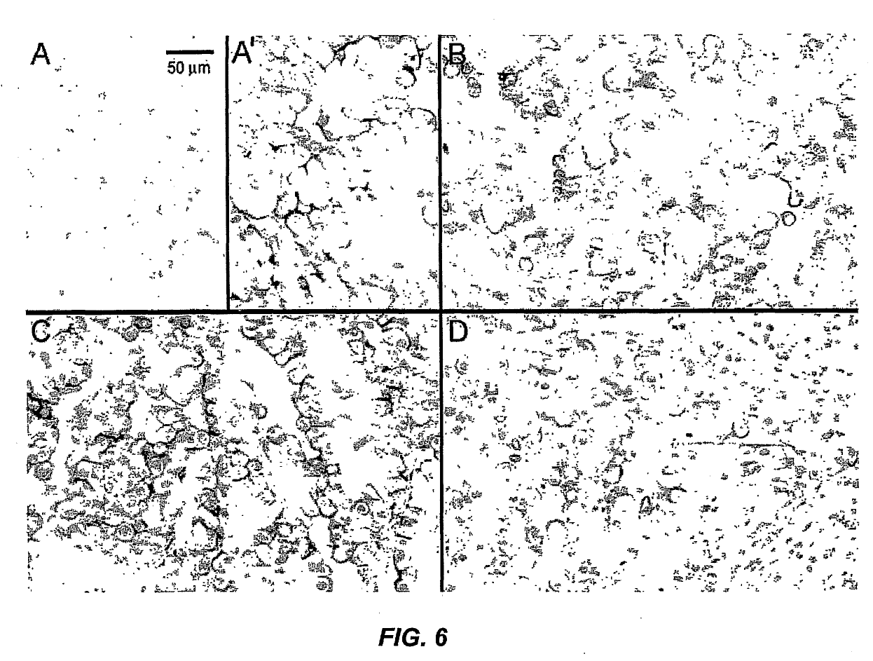

### DETAILED DESCRIPTION OF THE INVENTION

### I. Introduction

As noted in the Background, the currently available antibodies that specifically bind to mesothelin have not proved broadly useful for immunohistochemistry or immunoassays for mesothelin. For example, antibody K1, the first monoclonal antibody found that specifically bound mesothelin, does not work well on fixed tissue samples and did not detect mesothelin in lung cancer samples, while the commercially available anti-mesothelin antibody NCL-MESO (Novocastra Laboratories Ltd., Newcastle upon Tyne, U.K.), also referred to as the “5B2” antibody from the clone from which it is secreted, reacts with mesothelin expressed in bacteria, but does not react well with native mesothelin made in human cells. Surprisingly, the present invention provides antibodies that are particularly useful reagents for use in immunological studies and for detection of native mesothelin in human tissues.

The studies underlying the present invention used mesothelin-knockout mice. The mice were immunized with DNA encoding mesothelin and then boosted with injection of recombinant mesothelin protein. Mice with particularly high titers of antibodies were sacrificed, and a number of antibodies produced by the mice were studied. Two antibodies, designated as MN and MB, were found to have a surprising combination of both high affinity for mesothelin and utility in a range of immunohistochemical and immunological techniques, including staining fixed samples of tissue, labeling cells for FACS, labeling for Western blotting, and in labeling in ELISAs. For example, when compared against the K1 and 5B2 antibodies, the MN and MB antibodies were more than 500 times more reactive in ELISAs and more than 12 times more sensitive when used in Western blots, while having the same order of magnitude of sensitivity when used in FACs or immunohistochemical studies. See, Table 1, infra. Thus, the MN and MB are expected to have a range of applications in in vitro and ex vivo methods of detecting the presence of cells expressing mesothelin on their surfaces.

The MN and MB antibodies can, for example, be incorporated into chimeric immunoconjugates bearing a detectable label such as a radioisotope, a fluorescent moiety, or a reporter enzyme. These labeled immunoconjugates be used, for example, in in vitro assays to detect the presence of mesothelin-expressing cells in a biological sample.

The MN and MB antibodies have very high affinity for mesothelin. As shown in the Examples, MN has a dissociation constant (Kd) of 1.0 nM, while MB has a Kd of 0.6 nM. These binding affinities rival that of the anti-mesothelin antibody SS1, which has the highest affinity to mesothelin that was previously reported, and which is being used as the targeting portion of an immunotoxin currently in clinical trials for treatment of mesothelin-positive tumors. Thus, in addition to the use of MN and MB as reagents for immunohistochemistry and the like, it is expected that the intact MN and MB antibodies can also be used to target effector molecules, such as radionuclides chemically conjugated to the antibody, to cells which display mesothelin on their exterior surface. Further, the antibodies can enzymatically digested to create fragments, such as Fabs, that retain antigen recognition that can be used as the targeting portion of immunoconjugates. Alternatively, the Fv regions of the antibodies can be recombinantly produced in frame with a toxin moiety to produce the chimeric molecules known as immunotoxins. Typically, immunotoxins for treatment of solid tumors use single chain Fv regions (“scFvs”) or disulfide stabilized Fv regions (“dsFvs”) since the Fv regions are significantly smaller than whole immunoglobulins, which permits the immunotoxin to better penetrate into the tumor.

When made into immunotoxins, an immunotoxin made with the MN antibody was quite active. It is expected that immunotoxins made using the MN antibody in particular can be used in vitro or ex vivo to, for example, purge mesothelin-expressing cells in a cell culture. It is further expected that, like SS1, the MN antibody can be used in vivo to target therapeutic molecules, such as drugs, liposomes loaded with a drug, radionuclides, or, more preferably, cytotoxins, to mesothelin-expressing cells. For example, immunotoxins made with the MN antibodies of the invention can be used to inhibit the growth and proliferation of cancer cells bearing the mesothelin antigen.

Antibody MB binds a different epitope of mesothelin than that bound by antibody SS1 and by antibody MN. The combination of the MB antibody with an antibody that binds the epitope bound by SS1 or MN, therefore, can be used in sandwich assays and other immunological procedures in which the use of two antibodies binding to different epitopes is useful or desirable. In another embodiment, the MB antibody can be used in conjunction with a second antibody, such as MN or SS1, to form a bispecific antibody that will bind mesothelin at more than one epitope. In other embodiments, as discussed above, the MB antibody can be used by itself as the targeting portion of a chimeric molecule to target detectable labels or therapeutic agents, such as radioisotopes, to cells expressing mesothelin.

The MN and MB antibodies can be modified without changing their ability to be used for the purposes described above. As an initial matter, it is noted that the antibodies originated from mice immunized with mesothelin DNA and then boosted with mesothelin. The antibodies thus have framework regions (regions outside the complementarity determining regions, or “CDRs”) which contain the amino acid residues usually found in the framework regions in murine antibodies, and which may be immunogenic when administered to a human patient. To reduce immunogenicity of murine antibodies when used in humans, it is common in the art to engineer the framework regions by replacing residues found at particular positions in the antibodies of mice with the residues more typically found at the same position in human antibodies. Antibodies engineered in these ways are referred to as “humanized antibodies” and are typically preferred for in vivo use, since they have a lower risk of inducing side effects and typically can remain in the circulation longer. Methods of humanizing antibodies are known in the art and are set forth in some detail in, for example, U.S. Pat. Nos. 6,180,377; 6,407,213; 5,693,762; 5,585,089; and 5,530,101.

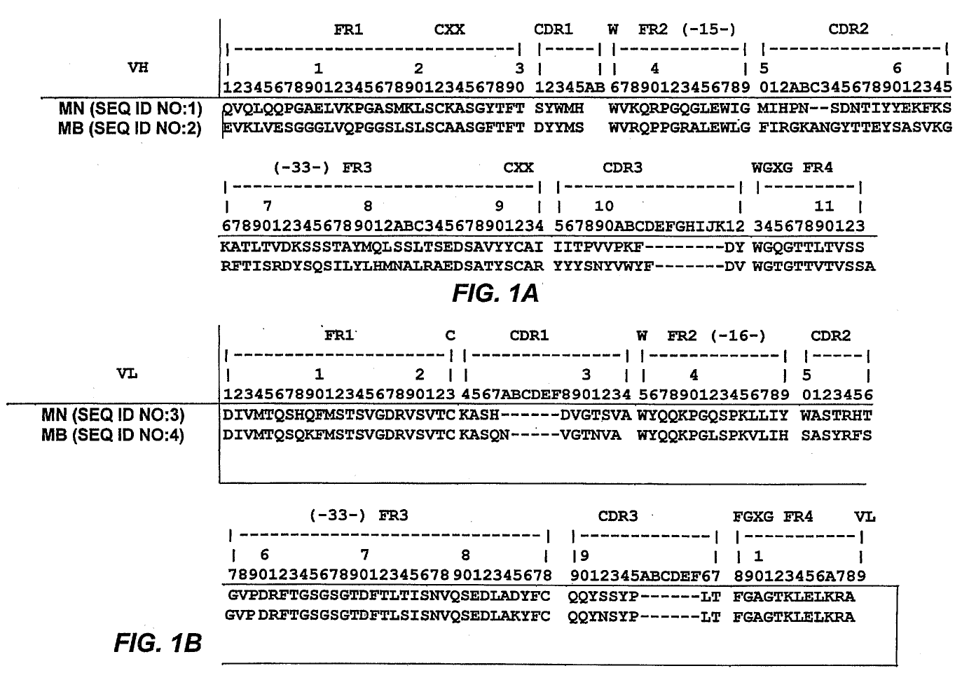

Further, since the CDRs of the variable regions determine antibody specificity, the CDRs set forth in FIG. 1 can be grafted or engineered into an antibody of choice to confer specificity for mesothelin upon that antibody. For example, the complementarity determining regions (CDRs), i.e., the antigen binding loops from the MN or MB antibody can be grafted onto a human antibody framework of known three dimensional structure (see, e.g., WO98/45322; WO 87/02671; U.S. Pat. Nos. 5,859,205; 5,585,089; and 4,816,567; EP Patent Application 0173494; Jones, et al. Nature 321:522 (1986); Verhoeyen, et al., Science 239:1534 (1988), Riechmann, et al. Nature 332:323 (1988); and Winter & Milstein, Nature 349:293 (1991)) to form an anti-mesothelin antibody with reduced or no immunogenic response when administered to humans.

The MN variable light chain (V_{L}) and variable heavy chain (V_{H}) sequences are shown in FIG. 1, which also sets forth the sequences of CDRs 1, 2, and 3 of each chain. The hybridoma secreting the antibody, MB-G-10, was deposited with the American Type Culture Collection (“ATCC”, Manassas, Va.) on May 11, 2005, under the terms of the Budapest Treaty. The ATCC accorded the hybridoma Patent Deposit Designation PTA-6709. The sequences of the V_{L} and V_{H} chains of the MB antibody, and of the CDRs of each chain, can be determined by comparison to the sequence of the antibody secreted by the deposited hybridoma.

The V_{L} and V_{H} chains of each antibody can be modified by engineering cysteines into the sequence to facilitate formation of disulfide bonds between the chains of the respective antibodies. A light chain and heavy chain of the variable region of an antibody joined by a disulfide bond between cysteines engineered into the framework region is known as a disulfide-stabilized Fv, or “dsFv.” Formation of dsFvs is taught in, for example, Pastan, U.S. Pat. No. 6,558,672, which sets forth a series of positions at which cysteines can be engineered into the framework region to facilitate formation of disulfide bonding between the chains, as well as in FitzGerald et al., International Publication Number WO 98/41641. Materials and Methods for constructing dsFvs are set forth in, for example, Kreitman et al., Clin. Cancer Res 6:1476-1487 (2000) and Kreitman et al., Intl J Cancer 81:148-155 (1999).

These methods can be used for generation of dsFvs of the MN and MB antibodies. Typically, the two chains are expressed from separate plasmids in inclusion bodies in a prokaryotic host cell, such as E. coli, and allowed to bond before the protein is purified from the inclusion bodies.

The antibodies of the present invention can also be used to form “chimeric antibodies” comprising the variable domains of the antibodies. The term “chimeric antibody” is used in the art to refer to an engineered antibody construct comprising variable domains of one species (such as mouse, rat, goat, sheep, cow, llama or camel variable domains), which may be humanized or not, and constant domains of another species (such as non-human primate or human constant domains) (for review see Hurle and Gross, Curr. Opin. Biotech. 5:428-433 (1994)). It should be clear that any method known in the art to develop chimeric antibodies or antibody constructs can be used. The present invention also concerns a diabody comprising a variable domain (including one which has been humanized) of an antibody of the invention. The term “diabody” relates to two non-covalently-linked scFv's, which then form a so-called diabody, as described in detail by Holliger et al. Proc. Natl. Acad. Sci. USA 90:6444 (1993) and reviewed by Poljak Structure 2:1121-1123 (1994). It should be clear that any method to generate diabodies, as for example described by these references and by Zhu et al. Biotechnology 14:192-196 (1996), can be used. Because of the multiplicity of forms in which the variable regions of the MN and the MB antibodies can be expressed, and to the variants of the antibodies which can be made, for convenience of reference, the discussion herein will sometimes refer to “MN antibodies” or “MB antibodies”.

The CDRs of the MN and MB antibodies can also be modified to improve their affinity. Work from the laboratory of the present inventors has established that the affinity of antibodies can be improved by mutating residues encoded by codons in mutational “hotspots,” which are nucleotide sequences where mutations are frequently concentrated during the in vivo affinity maturation process. Mutation of residues encoded by a codon with nucleotides within one of two consensus sequences is particularly useful. The two consensus sequences are (1) a tetranucleotide A/G-G-C/T-A/T (Pu-G-Py-A/T), and the serine codons AGY, where Y can be a C or a T (see, Wagner et al., Nature, 376:732 (1995); and Goyenechea and Milstein, Proc. Natl. Acad. Sci. USA 93:13979-13984 (1996)). The technique for mutating hotspots and selecting antibodies with increased affinity compared to the starting antibody (sometimes called the “parental” antibody) is explained in detail in, for example, PCT/US00/14829, International Publication No. WO 00/73346. Thus, it is contemplated that the affinity of the MN or the MB antibody, or both, can be improved by mutating residues in their CDRs, which residues are encoded by codons in one of the two consensus hotspot motifs set forth above. For convenience of reference, such residues can be referred to as “hot spot residues”.

It is also noted that making a conservative substitution of a CDR residue encoded by a codon whose nucleotides are not within a hot spot motif can often be made without markedly changing the affinity of the resulting antibody (for convenience, such a residue can be referred to as a “non-hot spot residue”). Persons of skill will therefore recognize that antibodies having a CDR with, for example, a single non-hot spot residue mutation compared to the CDRs set forth herein for the MN or the MB antibody, which have affinities close to those reported for the MN or the MB antibody, and which have similar efficacy in immunological assays and immunohistochemical techniques, can be used in the methods of the invention. For purposes of determining whether an antibody has an affinity close to that reported for the MN or the MB antibody, an antibody having CDRs which have the sequence of those set forth in FIG. 1 but in which one or more CDRs have a single non-hot spot residue mutation can be considered to have an affinity close to that reported for the MN or the MB antibody if its affinity is within 1 nM of that reported herein for the corresponding antibody (e.g., to that of the MN antibody if the CDRs are those of the MN antibody except for the mutation of the non-hot spot residue and, optionally, of a hot spot residue). For purposes of determining whether an antibody has similar efficacy in immunological assays and immunohistochemical techniques to that reported herein for the MN or the MB antibody, an antibody having CDRs which have the sequence of those set forth in FIG. 1 but in which one or more CDRs have a single non-hot spot residue mutation can be considered to have an affinity close to that reported for the MN or the MB antibody if it's affinity is within 1 nM of that reported for the corresponding antibody.

As described above, therefore, it is contemplated that the MN and MB antibodies can be modified in various ways without losing antigen recognition capability. Thus, the invention provides antibodies which specifically bind mesothelin and which have V_{H} chains with at least 90% sequence identity to the sequence of the V_{H} chain of the MN antibody (SEQ ID NO:1) and V_{L} chains with at least 90% sequence identity to the sequence of the V_{L} chain of the MN antibody (SEQ ID NO:3) or which have V_{H} chains with at least 90% sequence identity to the sequence of the V_{H} chain of the MB antibody and V_{L} chains with at least 90% sequence identity to the sequence of the V_{L} chain of the MB antibody

In more preferred embodiments, the invention provides antibodies which specifically bind mesothelin and which have V_{H} chains with at least 95% sequence identity to the sequence of the V_{H} chain of the MN antibody (SEQ ID NO:1) and V_{L} chains with at least 95% sequence identity to the sequence of the V_{L} chain of the MN antibody (SEQ ID NO:3) or which have V_{H} chains with at least 95% sequence identity to the sequence of the V_{H} chain of the MB antibody (Patent Deposit Designation PTA-6709) and V_{L} chains with at least 95% sequence identity to the sequence of the V_{L} chain of the MB antibody (Patent Deposit Designation PTA-6709). Preferably, the antibodies have a Kd with respect to mesothelin of at least 10 nM, and more preferably of at least 5 nM when affinity is measured as set forth in the Examples. Further, it is preferable that the antibodies retain the activity of the MN antibody or of the MB antibody for immunohistochemical staining of fixed tissue and immunological techniques such as FACS, Western blotting and ELISAs. Whether or not a modified antibody retains this utility can be readily determined by, for example, conducting one of these tests with the modified antibody and comparing the results to the results of a like test conducted using the MN or the MB antibody.

In some embodiments, the antibodies have V_{H} chains with at least 96%, 97%, 98%, or even higher sequence identity to the sequence of the V_{H} chain of the MN antibody (SEQ ID NO:1) and V_{L} chains with at least 96%, 97%, 98%, or even higher sequence identity to the sequence of the V_{L} chain of the MN antibody (SEQ ID NO:3) or which have V_{H} chains with at least 96%, 97%, 98%, or even higher sequence identity to the sequence of the V_{H} chain of the MB antibody (ATCC Patent Deposit Designation PTA-6709) and V_{L} chains with at least 96%, 97%, 98%, or even higher sequence identity to the sequence of the V_{L} chain of the MB antibody (ATCC Patent Deposit Designation PTA-6709), and which retain specific binding to mesothelin with a Kd of at least 10 nM, and more preferably of at least 5 nM when affinity is measured as set forth in the Examples. Additionally, it is preferable that the antibodies retain utility in immunohistochemical and immunological techniques such as Western blots and ELISAs. Utility in these techniques is easily measured, as shown in the Examples.

In some embodiments, the antibodies have CDRs with the sequences set forth in FIG. 1. In some embodiments, the antibodies have (a) CDRs which have the sequences set forth in FIG. 1 for antibody MN or of the deposited MB antibody, except for one or more mutations of residues encoded by a codon with nucleotides within a consensus sequence selected from A/G-G-C/T-A/T (Pu-G-Py-A/T), and AGY, where Y can be a C or a T, and (b) the same or greater affinity for mesothelin than the starting MN or MB antibody. In some embodiments, the antibodies have (a) CDRs which have the sequences set forth in FIG. 1 for antibody MN or of the deposited MB antibody, except that one or more of the CDRs have one mutation of residues encoded by a codon with nucleotides that do not fall within a consensus sequence, (b) an affinity for mesothelin that is similar to that of the MN or MB antibody and (c) similar efficacy when used for immunoassays or immunohistochemical techniques. In some embodiments, the antibodies have (a) CDRs which have the sequences set forth in FIG. 1 except for one or more mutations of residues encoded by a codon with nucleotides within a consensus sequence selected from A/G-G-C/T-A/T (Pu-G-Py-A/T), and AGY, where Y can be a C or a T, (b) one mutation of a residue encoded by a codon with nucleotides that do not fall within a consensus sequence, (c) an affinity for mesothelin that is similar to that of the MN or MB antibody and, (d) similar efficacy to that of the MN or MB antibody when used for immunoassays or immunohistochemical techniques.

It is expected that some of the antibodies made by mutating residues in hot spots in the CDRs of the MN or the MB antibodies will have affinities higher than that of the starting antibody. It is not expected that the affinity of these yet-higher affinity antibodies will reach zero, which would reflect a covalent bond between the antibody and the antigen. The affinities of the MN antibody and of the MB antibody are quite good: the affinity of the MN antibody is 1 nM while the affinity of the MB antibody is 0.6 nM. It is therefore expected that forms of these antibodies in which hot spot residues are mutated can be expected to have affinities stated in tenths of a nM. For purposes of being able to state a lower limit on the affinity on the mutated antibodies, the limit may be stated as 0.05 nM.

In some embodiments, the invention provides antibodies which have CDRs 1, 2, and 3 of the V_{H} and CDRs 1, 2, and 3 of the V_{L} chain of the MN antibody as those CDRs are shown in FIG. 1, or which have CDRs 1, 2, and 3 of the V_{H} and CDRs 1, 2, and 3 of the V_{L} chain of the deposited MB antibody (for convenience of reference, the comparison of the sequence of CDRs 1, 2, and 3 of the V_{H} and CDRs 1, 2, and 3 of the V_{L} chain of the antibodies under study to those of the deposited MB antibody will be to the “CDRs of the corresponding chain of the MB antibody”). Preferably, the antibodies of the invention have the sequences of SEQ ID NOS:1 and 3, or of the MB antibody secreted by the MB hybridoma. In some preferred forms, the two chains will be linked by a peptide linker, to form a scFv, or may have one or more cysteine residues engineered into the framework region to permit formation of a disulfide bond linking the two chains together.

The sequences of V_{H} and V_{L} chains comprising CDRs 1, 2, and 3 of the V_{H} and CDRs 1, 2, and 3 of the V_{L} chain of the MN antibody or which have CDRs 1, 2, and 3 of the V_{H} and CDRs 1, 2, and 3 of the V_{L} chain of the MB antibody, can also be used as the Fv regions of intact immunoglobulins. Persons of skill are aware that the Fc region of antibodies of different classes, or isotypes (IgG, IgA, IgM, etc.), is relatively invariant, and that the specificity of, for example, an IgG molecule, can be altered by engineering into the IgG a selected Fv region. Accordingly, by grafting onto the Fc region an Fv region or Fv regions of the invention (such as those comprising CDRs 1, 2, and 3 of the V_{H} and CDRs 1, 2, and 3 of the V_{L} chain of the MN antibody or which have CDRs 1, 2, and 3 of the V_{H} and CDRs 1, 2, and 3 of the V_{L} chain of the MB antibody), specificity and affinity for mesothelin can be conferred to the immunoglobulin molecule.

In general, even if intact immunoglobulins are made using Fvs of the invention, use of fragments of the intact immunoglobulins that retain antigen recognition, such as an Fab, an Fab′, a scFv, a dsFv, or a diabody, is preferred. Many of the recombinant immunotoxins produced from constructs of scFv are one-third the size of IgG-toxin chemical conjugates and are homogeneous in composition. Elimination of the constant portion of the IgG molecule from the scFv results in faster clearance of the immunotoxin after injection into animals, including primates, and the smaller size of the conjugates improves drug penetration in solid tumors. Together, these properties lessen the side effects associated with the toxic moiety by reducing the time in which the immunotoxin (IT) interacts with non-target tissues and tissues that express very low levels of antigen.

These advantages, however, are offset to some degree by the loss of antigen binding affinity that occurs when IgGs, for example, are converted to scFvs (Reiter et al., Nature Biotechnol. 14:239-1245 (1996)). Increasing affinity has been shown to improve selective tumor delivery of scFvs (Adams et al., Cancer Res. 58:485-490 (1998)), and is likely to increase their usefulness in tumor imaging and treatment. The affinity of the antibodies of the invention, however, is so high that it is expected that agents based on these antibodies will be effective in delivering effector molecules to their intended targets. The high affinity of the antibodies of the invention is therefore important and provides an alternative to the use of the SS1 antibody and other high affinity anti-mesothelin antibodies for delivering agents to cells expressing mesothelin, providing the practitioner with more flexibility in the choice of targeting moieties in fashioning immunoconjugates.

### II. Definitions

Units, prefixes, and symbols are denoted in their Systéme International de Unites (SI) accepted form. Numeric ranges are inclusive of the numbers defining the range. Unless otherwise indicated, nucleic acids are written left to right in 5′ to 3′ orientation; amino acid sequences are written left to right in amino to carboxy orientation. The headings provided herein are not limitations of the various aspects or embodiments of the invention, which can be had by reference to the specification as a whole. Accordingly, the terms defined immediately below are more fully defined by reference to the specification in its entirety.

The term “mesothelin” refers to a protein and fragments thereof present on the surface of some human cells and bound by, for example, the K1 antibody. The preferred nucleic acid and amino acid sequences of mesothelin are set forth in, for example, PCT published application WO 97/25,068 and U.S. Pat. Nos. 6,083,502 and 6,153,430. See also, Chang, K. & Pastan, I., Int. J. Cancer 57:90 (1994); Chang, K. & Pastan, I., Proc. Nat'l Acad. Sci. USA 93:136 (1996); Brinkmann U., et al., Int. J. Cancer 71:638 (1997); Chowdhury, P. S., et al., Mol. Immunol. 34:9 (1997), and U.S. Pat. No. 6,809,184. Mesothelin is expressed as a precursor protein of approximately 69 kDa, that then is processed to release a 30 kDa protein, while leaving attached to the cell surface the 40 kDa glycosylphosphatidylinositol linked cell surface glycoprotein described in the Background. The 40 kDa glycoprotein is the one referred to by the term “mesothelin” herein.

The antibodies designated MN and MB are murine antibodies produced by hybridomas. The sequence of the Fv region of the MN antibody is described herein. The hybridoma secreting the MB antibody, designated hybridoma MB-G-10, was deposited with the ATCC on May 11, 2005, under the terms of the Budapest Treaty. The ATCC has accorded the hybridoma Patent Deposit Designation PTA-6709.

“Antibodies” exist as intact immunoglobulins or as a number of well characterized fragments produced by digestion with various peptidases. Thus, for example, pepsin digests an antibody below the disulfide linkages in the hinge region to produce F(ab)′₂, a dimer of Fab which itself is a light chain joined to VH—CH by a disulfide bond. The F(ab)′₂ may be reduced under mild conditions to break the disulfide linkage in the hinge region thereby converting the (Fab′)₂ dimer into a Fab′ monomer. The Fab′ monomer is essentially a Fab with part of the hinge region (see, W. E. Paul, ed., Fundamental Immunology, Raven Press, N.Y. (1993), for a more detailed description of these and other antibody fragments). While various antibody fragments are defined in terms of the digestion of an intact antibody, one of skill will appreciate that such Fab′ fragments may be synthesized de novo either chemically or by utilizing recombinant DNA methodology.

For convenience of reference, as used herein, the term “antibody” includes whole (sometimes referred to herein as “intact”) antibodies, antibody fragments that retain antigen recognition and binding capability, whether produced by the modification of whole antibodies or synthesized de novo using recombinant DNA methodologies, monoclonal antibodies, polyclonal antibodies, and antibody mimics, unless otherwise required by context. The antibody may be an IgM, IgG (e.g. IgG₁, IgG₂, IgG₃ or IgG₄), IgD, IgA or IgE).

The term “antibody fragments” means molecules that comprise a portion of an intact antibody, generally the antigen binding or variable region of the intact antibody. Examples of antibody fragments include Fab, Fab′, F(ab′)2, and Fv fragments; helix-stabilized antibodies (see, e.g., Arndt et al., J Mol Biol 312:221-228 (2001); diabodies (see below); single-chain antibody molecules (“scFvs,” see, e.g., U.S. Pat. No. 5,888,773); disulfide stabilized antibodies (“dsFvs”, see, e.g., U.S. Pat. Nos. 5,747,654 and 6,558,672), and domain antibodies (“dAbs,” see, e.g., Holt et al., Trends Biotech 21(11):484-490 (2003), Ghahroudi et al., FEBS Lett. 414:521-526 (1997), Lauwereys et al., EMBO J. 17:3512-3520 (1998), Reiter et al., J. Mol. Biol. 290:685-698 (1999), Davies and Riechmann, Biotechnology, 13:475-479 (2001)).

As used herein, the term “anti-mesothelin” in reference to an antibody, includes reference to an antibody which is generated against mesothelin. In preferred embodiments, the mesothelin is a primate mesothelin such as human mesothelin. In a particularly preferred embodiment, the antibody is generated against human mesothelin synthesized by a non-primate mammal after introduction into the animal of cDNA which encodes human mesothelin.

The term “diabodies” refers to small antibody fragments with two antigen-binding sites, which fragments comprise a variable heavy domain (“V_{H}” or “VH”) connected to a variable light domain (“V_{L}” or “VL”) in the same polypeptide chain (V_{H}-V_{L}). By using a linker that is too short to allow pairing between the two domains on the same chain, the domains are forced to pair with the complementary domains of another chain and create two antigen-binding sites. Diabodies and their production are described more fully in, for example, EP 404,097; WO 93/11161; and Holliger et al., Proc. Natl. Acad. Sci. USA, 90: 6444-6448 (1993).

Typically, an immunoglobulin has a heavy and light chain. Each heavy and light chain contains a constant region and a variable region, (the regions are also known as “domains”). Light and heavy chain variable regions contain a “framework” region interrupted by three hypervariable regions, also called “complementarity-determining regions” or “CDRs”. The extent of the framework region and CDRs have been defined. (see, Kabat, E., et al., SEQUENCES OF PROTEINS OF IMMUNOLOGICAL INTEREST, U.S. Department of Health and Human Services, (1987), which is hereby incorporated by reference). The sequences of the framework regions of different light or heavy chains are relatively conserved within a species. The framework region of an antibody, that is the combined framework regions of the constituent light and heavy chains, serves to position and align the CDRs in three dimensional space.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a VH CDR3 is located in the variable domain of the heavy chain of the antibody in which it is found, whereas a VL CDR1 is the CDR1 from the variable domain of the light chain of the antibody in which it is found.

References to “VH” or a “V_{H}” refer to the variable region of an immunoglobulin heavy chain, including an Fv, scFv, dAb, dsFv or Fab. References to “VL” or a “V_{L}” refer to the variable region of an immunoglobulin light chain, including of an Fv, scFv, dsFv, dAb, or Fab.

The term “Fv” refers to the variable domains of the heavy chain and of the light chain of an antibody. The phrase “single chain Fv” or “scFv” refers to an antibody in which the variable domains of the heavy chain and of the light chain of a traditional two chain antibody have been joined to form one chain. Optionally, a linker (usually a peptide) is inserted between the two chains to allow for proper folding and creation of an active binding site. If a linker is present, it is excluded for purposes of comparing the percentage of sequence identity between a given VH or VL chain and a VH or VL chain of the MN or the MB antibodies.

An antibody immunologically reactive with a particular antigen can be generated by recombinant methods such as selection of libraries of recombinant antibodies in phage or similar vectors, see, e.g., Huse, et al., Science 246:1275-1281 (1989); Ward, et al., Nature 341:544-546 (1989); and Vaughan, et al., Nature Biotech. 14:309-314 (1996), or by immunizing an animal with the antigen or with DNA encoding the antigen.

The extent of the framework region and CDRs have been defined. The sequences of the framework regions of different light or heavy chains are relatively conserved within a species. The framework region of an antibody, that is the combined framework regions of the constituent light and heavy chains, serves to position and align the CDRs in three dimensional space.

The term “linker peptide” includes reference to a peptide within an antibody binding fragment (e.g., Fv fragment) which serves to indirectly bond the variable domain of the heavy chain to the variable domain of the light chain.

The term “parental antibody” means any antibody of interest which is to be mutated or varied to obtain antibodies or fragments thereof which bind to the same epitope as the parental antibody, but with higher affinity.

The term “hotspot” means a portion of a nucleotide sequence of a CDR or of a framework region of a variable domain which is a site of particularly high natural variation. Although CDRs are themselves considered to be regions of hypervariability, it has been learned that mutations are not evenly distributed throughout the CDRs. Particular sites, or hotspots, have been identified as these locations which undergo concentrated mutations. The hotspots are characterized by a number of structural features and sequences. These “hotspot motifs” can be used to identify hotspots. Two consensus sequences motifs which are especially well characterized are the tetranucleotide sequence RGYW and the serine sequence AGY, where R is A or G, Y is C or T, and W is A or T.

A “targeting moiety” is the portion of an immunoconjugate intended to target the immunoconjugate to a cell of interest. Typically, the targeting moiety is an antibody, a scFv, a dsFv, an Fab, or an F(ab′)₂.

A “toxic moiety” is the portion of a immunotoxin which renders the immunotoxin cytotoxic to cells of interest.

A “therapeutic moiety” is the portion of an immunoconjugate intended to act as a therapeutic agent.

The term “therapeutic agent” includes any number of compounds currently known or later developed to act as anti-neoplastics, anti-inflammatories, cytokines, anti-infectives, enzyme activators or inhibitors, allosteric modifiers, antibiotics or other agents administered to induce a desired therapeutic effect in a patient. The therapeutic agent may also be a toxin or a radioisotope, where the therapeutic effect intended is, for example, the killing of a cancer cell.

A “detectable label” means, with respect to an immunoconjugate, a portion of the immunoconjugate which has a property rendering its presence detectable. For example, the immunoconjugate may be labeled with a radioactive isotope which permits cells in which the immunoconjugate is present to be detected in immunohistochemical assays.

The term “effector moiety” means the portion of an immunoconjugate intended to have an effect on a cell targeted by the targeting moiety or to identify the presence of the immunoconjugate. Thus, the effector moiety can be, for example, a therapeutic moiety, a toxin, a radiolabel, or a fluorescent label.

The terms “chimeric molecule” and “immunoconjugate” refer to linkage of an antibody to an effector moiety. The linkage is usually a covalent bond between the effector moiety and the antibody. The linkage can be by chemical conjugation, or by expressing the antibody and the effector moiety from a nucleic acid encoding both the antibody and the effector moiety. For example, a nucleic acid encoding an MN or MB antibody of the invention fused to a Pseudomonas exotoxin can be recombinantly expressed in E. coli and then isolated.

The terms “effective amount” or “amount effective to” or “therapeutically effective amount” includes reference to a dosage of a therapeutic agent sufficient to produce a desired result, such as inhibiting cell protein synthesis by at least 50%, or killing the cell.

The term “toxin” includes reference to abrin, ricin, Pseudomonas exotoxin (PE), diphtheria toxin (DT), botulinum toxin, or modified toxins thereof. For example, PE and DT are highly toxic compounds that typically bring about death through liver toxicity. PE and DT, however, can be modified into a form for use as an immunotoxin by removing the native targeting component of the toxin (e.g., domain Ia of PE or the B chain of DT) and replacing it with a different targeting moiety, such as an antibody.

The term “connected to,” in relation to an antibody and a therapeutic moiety or detectable label, means that the antibody is fused to (e.g., by recombinant expression) or conjugated to (e.g., chemically attached to) the therapeutic moiety or detectable label, directly or through a linker.

The term “contacting” includes reference to placement in direct physical association.

An “expression plasmid” comprises a nucleotide sequence encoding a molecule or interest, which is operably linked to a promoter.

As used herein, “polypeptide”, “peptide” and “protein” are used interchangeably and include reference to a polymer of amino acid residues. The terms apply to amino acid polymers in which one or more amino acid residue is an artificial chemical analogue of a corresponding naturally occurring amino acid, as well as to naturally occurring amino acid polymers. The terms also apply to polymers containing conservative amino acid substitutions such that the protein remains functional.

The term “residue” or “amino acid residue” or “amino acid” includes reference to an amino acid that is incorporated into a protein, polypeptide, or peptide (collectively “peptide”). The amino acid can be a naturally occurring amino acid and, unless otherwise limited, can encompass known analogs of natural amino acids that can function in a similar manner as naturally occurring amino acids.

The amino acids and analogs referred to herein are described by shorthand designations as follows in Table A:

TABLE A
Amino Acid Nomenclature

| Name | 3-letter | 1-letter |
|---|---|---|
| Alanine | Ala | A |
| Arginine | Arg | R |
| Asparagine | Asn | N |
| Aspartic Acid | Asp | D |
| Cysteine | Cys | C |
| Glutamic Acid | Glu | E |
| Glutamine | Gln | Q |
| Glycine | Gly | G |
| Histidine | His | H |
| Homoserine | Hse | — |
| Isoleucine | Ile | I |
| Leucine | Leu | L |
| Lysine | Lys | K |
| Methionine | Met | M |
| Methionine sulfoxide | Met (O) | — |
| Methionine | Met (S-Me) | — |
| methylsulfonium | | |
| Norleucine | Nle | — |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

A “conservative substitution”, when describing a protein refers to a change in the amino acid composition of the protein that does not substantially alter the protein's activity. Thus, “conservatively modified variations” of a particular amino acid sequence refers to amino acid substitutions of those amino acids that are not critical for protein activity or substitution of amino acids with other amino acids having similar properties (e.g., acidic, basic, positively or negatively charged, polar or non-polar, etc.) such that the substitutions of even critical amino acids do not substantially alter activity. Conservative substitution tables providing functionally similar amino acids are well known in the art. The following six groups in Table B each contain amino acids that are conservative substitutions for one another:

TABLE B

1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).
See also, Creighton, Proteins: Structures and Molecular Properties, W. H. Freeman and Company, New York (2nd Ed., 1992).

The terms “substantially similar” in the context of a peptide indicates that a peptide comprises a sequence with at least 90%, preferably at least 95% sequence identity to the reference sequence over a comparison window of 10-20 amino acids. Percentage of sequence identity is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The phrase “disulfide bond” or “cysteine-cysteine disulfide bond” refers to a covalent interaction between two cysteines in which the sulfur atoms of the cysteines are oxidized to form a disulfide bond. The average bond energy of a disulfide bond is about 60 kcal/mol compared to 1-2 kcal/mol for a hydrogen bond. In the context of this invention, the cysteines which form the disulfide bond are within the framework regions of the single chain antibody and serve to stabilize the conformation of the antibody.

The terms “conjugating,” “joining,” “bonding” or “linking” refer to making two polypeptides into one contiguous polypeptide molecule. In the context of the present invention, the terms include reference to joining an antibody moiety to an effector molecule (EM). The linkage can be either by chemical or recombinant means. Chemical means refers to a reaction between the antibody moiety and the effector molecule such that there is a covalent bond formed between the two molecules to form one molecule.

As used herein, “recombinant” includes reference to a protein produced using cells that do not have, in their native state, an endogenous copy of the DNA able to express the protein. The cells produce the recombinant protein because they have been genetically altered by the introduction of the appropriate isolated nucleic acid sequence. The term also includes reference to a cell, or nucleic acid, or vector, that has been modified by the introduction of a heterologous nucleic acid or the alteration of a native nucleic acid to a form not native to that cell, or that the cell is derived from a cell so modified. Thus, for example, recombinant cells express genes that are not found within the native (non-recombinant) form of the cell, express mutants of genes that are found within the native form, or express native genes that are otherwise abnormally expressed, underexpressed or not expressed at all.

As used herein, “nucleic acid” or “nucleic acid sequence” includes reference to a deoxyribonucleotide or ribonucleotide polymer in either single- or double-stranded form, and unless otherwise limited, encompasses known analogues of natural nucleotides that hybridize to nucleic acids in a manner similar to naturally occurring nucleotides. Unless otherwise indicated, a particular nucleic acid sequence includes the complementary sequence thereof as well as conservative variants, i.e., nucleic acids present in wobble positions of codons and variants that, when translated into a protein, result in a conservative substitution of an amino acid.

As used herein, “encoding” with respect to a specified nucleic acid, includes reference to nucleic acids which comprise the information for translation into the specified protein. The information is specified by the use of codons. Typically, the amino acid sequence is encoded by the nucleic acid using the “universal” genetic code. However, variants of the universal code, such as is present in some plant, animal, and fungal mitochondria, the bacterium Mycoplasma capricolumn (Proc. Nat'l Acad. Sci. USA 82:2306-2309 (1985), or the ciliate Macronucleus, may be used when the nucleic acid is expressed in using the translational machinery of these organisms.

The phrase “fusing in frame” refers to joining two or more nucleic acid sequences which encode polypeptides so that the joined nucleic acid sequence translates into a single chain protein which comprises the original polypeptide chains.

As used herein, “expressed” includes reference to translation of a nucleic acid into a protein. Proteins may be expressed and remain intracellular, become a component of the cell surface membrane or be secreted into the extracellular matrix or medium.

By “host cell” is meant a cell which can support the replication or expression of the expression vector. Host cells may be prokaryotic cells such as E. coli, or eukaryotic cells such as yeast, insect, amphibian, or mammalian cells.

The phrase “phage display library” refers to a population of bacteriophage, each of which contains a foreign cDNA recombinantly fused in frame to a surface protein. The phage display the foreign protein encoded by the cDNA on its surface. After replication in a bacterial host, typically E. coli, the phage which contain the foreign cDNA of interest are selected by the expression of the foreign protein on the phage surface.

The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence, as measured using one of the following sequence comparison algorithms or by visual inspection.

The phrase “substantially identical,” in the context of two nucleic acids or polypeptides, refers to two or more sequences or subsequences that have at least 60%, preferably 80%, most preferably 90-95% nucleotide or amino acid residue identity, when compared and aligned for maximum correspondence, as measured using one of the following sequence comparison algorithms or by visual inspection. Preferably, the substantial identity exists over a region of the sequences that is at least about 50 residues in length, more preferably over a region of at least about 100 residues, and most preferably the sequences are substantially identical over at least about 150 residues. In a most preferred embodiment, the sequences are substantially identical over the entire length of the coding regions.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215: 403-410 and Altschuel et al. (1977) Nucleic Acids Res. 25: 3389-3402, respectively. Software for performing BLAST analyses is publicly available through the National Center for Biotechnology Information (on the internet by entering “www.” followed by “ncbi.nlm.nih.gov/”). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions, as described below.

The term “in vivo” includes reference to inside the body of the organism from which the cell was obtained. “Ex vivo” and “in vitro” means outside the body of the organism from which the cell was obtained.

The phrase “malignant cell” or “malignancy” refers to tumors or tumor cells that are invasive and/or able to undergo metastasis, i.e., a cancerous cell.

As used herein, “mammalian cells” includes reference to cells derived from mammals including humans, rats, mice, guinea pigs, chimpanzees, or macaques. The cells may be cultured in vivo or in vitro.

The term “selectively reactive” refers, with respect to an antigen, the preferential association of an antibody, in whole or part, with a cell or tissue bearing that antigen and not to cells or tissues lacking that antigen. It is, of course, recognized that a certain degree of non-specific interaction may occur between a molecule and a non-target cell or tissue. Nevertheless, selective reactivity, may be distinguished as mediated through specific recognition of the antigen. Although selectively reactive antibodies bind antigen, they may do so with low affinity. On the other hand, specific binding results in a much stronger association between the antibody and cells bearing the antigen than between the bound antibody and cells lacking the antigen. Specific binding typically results in greater than 2-fold, preferably greater than 5-fold, more preferably greater than 10-fold and most preferably greater than 100-fold increase in amount of bound antibody (per unit time) to a cell or tissue bearing mesothelin as compared to a cell or tissue lacking mesothelin. Specific binding to a protein under such conditions requires an antibody that is selected for its specificity for a particular protein. A variety of immunoassay formats are appropriate for selecting antibodies specifically immunoreactive with a particular protein. For example, solid-phase ELISA immunoassays are routinely used to select monoclonal antibodies specifically immunoreactive with a protein. See Harlow & Lane, ANTIBODIES, A LABORATORY MANUAL, Cold Spring Harbor Publications, New York (1988), for a description of immunoassay formats and conditions that can be used to determine specific immunoreactivity.

The term “immunologically reactive conditions” includes reference to conditions which allow an antibody generated to a particular epitope to bind to that epitope to a detectably greater degree than, and/or to the substantial exclusion of, binding to substantially all other epitopes. Immunologically reactive conditions are dependent upon the format of the antibody binding reaction and typically are those utilized in immunoassay protocols or those conditions encountered in vivo. See Harlow & Lane, supra, for a description of immunoassay formats and conditions. Preferably, the immunologically reactive conditions employed in the methods of the present invention are “physiological conditions” which include reference to conditions (e.g., temperature, osmolarity, pH) that are typical inside a living mammal or a mammalian cell. While it is recognized that some organs are subject to extreme conditions, the intra-organismal and intracellular environment normally lies around pH 7 (i.e., from pH 6.0 to pH 8.0, more typically pH 6.5 to 7.5), contains water as the predominant solvent, and exists at a temperature above 0° C. and below 50° C. Osmolarity is within the range that is supportive of cell viability and proliferation.

### III. Anti-Mesothelin Antibodies

In preferred embodiments of the present invention, the anti-mesothelin antibody is a recombinant antibody such as a scFv or a disulfide stabilized Fv antibody. Fv antibodies are typically about 25 kDa and contain a complete antigen-binding site with 3 CDRs per heavy and light chain. If the V_{H} and the V_{L} chain are expressed non-contiguously, the chains of the Fv antibody are typically held together by noncovalent interactions. However, these chains tend to dissociate upon dilution, so methods have been developed to crosslink the chains through glutaraldehyde, intermolecular disulfides, or a peptide linker.

In a preferred embodiment, the antibody is a single chain Fv (scFv). The V_{H} and the V_{L} regions of a scFv antibody comprise a single chain which is folded to create an antigen binding site similar to that found in two chain antibodies. Once folded, noncovalent interactions stabilize the single chain antibody. In a more preferred embodiment, the scFv is recombinantly produced. In yet another preferred embodiment, the V_{H} and V_{L} regions have the amino acid sequences shown in FIG. 1. One of skill will realize that conservative variants of the antibodies of the instant invention can be made. Such conservative variants employed in scFv fragments will retain critical amino acid residues necessary for correct folding and stabilizing between the V_{H} and the V_{L} regions.

In some embodiments of the present invention, the scFv antibody is directly linked to the EM through the light chain. However, scFv antibodies can be linked to the EM via its amino or carboxyl terminus.

While the V_{H} and V_{L} regions of some antibody embodiments can be directly joined together, one of skill will appreciate that the regions may be separated by a peptide linker consisting of one or more amino acids. Peptide linkers and their use are well-known in the art. See, e.g., Huston, et al., Proc. Nat'l Acad. Sci. USA 8:5879 (1988); Bird, et al., Science 242:4236 (1988); Glockshuber, et al., Biochemistry 29:1362 (1990); U.S. Patent No. 4,946,778, U.S. Pat. No. 5,132,405 and Stemmer, et al., Biotechniques 14:256-265 (1993), all incorporated herein by reference. Generally the peptide linker will have no specific biological activity other than to join the regions or to preserve some minimum distance or other spatial relationship between them. However, the constituent amino acids of the peptide linker may be selected to influence some property of the molecule such as the folding, net charge, or hydrophobicity. Single chain Fv (scFv) antibodies optionally include a peptide linker of no more than 50 amino acids, generally no more than 40 amino acids, preferably no more than 30 amino acids, and more preferably no more than 20 amino acids in length. In some embodiments, the peptide linker is a concatemer of the sequence Gly-Gly-Gly-Ser, preferably 2, 3, 4, 5, or 6 such sequences. However, it is to be appreciated that some amino acid substitutions within the linker can be made. For example, a valine can be substituted for a glycine.

Methods of making scFv antibodies have been described. See, e.g., Ward, et al. Nature 341:544-546 (1989). In brief, mRNA from B-cells is isolated and cDNA is prepared. The cDNA is amplified by well known techniques, such as PCR, with primers specific for the variable regions of heavy and light chains of immunoglobulins. The PCR products are purified by, for example, agarose gel electrophoresis, and the nucleic acid sequences are joined. If a linker peptide is desired, nucleic acid sequences that encode the peptide are inserted between the heavy and light chain nucleic acid sequences. The sequences can be joined by techniques known in the art, such as blunt end ligation, insertion of restriction sites at the ends of the PCR products or by splicing by overlap extension (Chowdhury, et al., Mol. Immunol. 34:9 (1997)). After amplification, the nucleic acid which encodes the scFv is inserted into a vector, again by techniques well known in the art. Preferably, the vector is capable of replicating in prokaryotes and of being expressed in both eukaryotes and prokaryotes.

In a preferred embodiment, scFvs are chosen through a phage display library. The procedure described above for synthesizing scFv is followed. After amplification by PCR, the scFv nucleic acid sequences are fused in frame with gene III (gIII) which encodes the minor surface protein gIIIp of the filamentous phage (Marks, et al., J. Biol. Chem. 267:16007-16010 (1992); Marks, et al., Behring Inst. Mitt. 91:6-12 (1992); and Brinkmann, et al., J. Immunol. Methods 182:41-50 (1995)). The phage express the resulting fusion protein on their surface. Since the proteins on the surface of the phage are functional, phage bearing mesothelin-binding antibodies can be separated from non-binding or lower affinity phage by panning or antigen affinity chromatography (McCafferty, et al., Nature 348:552-554 (1990)).

scFv that specifically bind to mesothelin are typically found by panning. Panning is done by coating a solid surface with mesothelin and incubating the phage on the surface for a suitable time under suitable conditions. The unbound phage are washed off the solid surface and the bound phage are eluted. Finding the antibody with the highest affinity is dictated by the efficiency of the selection process and depends on the number of clones that can be screened and the stringency with which it is done. Typically, higher stringency corresponds to more selective panning. If the conditions are too stringent, however, the phage will not bind. After one round of panning, the phage that bind to mesothelin coated plates are expanded in E. coli and subjected to another round of panning. In this way, an enrichment of 2000-fold occurs in 3 rounds of panning. Thus, even when enrichment in each round is low, multiple rounds of panning will lead to the isolation of rare phage and the genetic material contained within which encodes the sequence of the highest affinity antibody. The physical link between genotype and phenotype provided by phage display makes it possible to test every member of a cDNA library for binding to antigen, even with large libraries of clones.

The antibodies of this invention bind to mesothelin with an affinity at least that of MN or of MB. Binding affinity for a target antigen is typically measured or determined by standard antibody-antigen assays, such as competitive assays, saturation assays, or immunoassays such as ELISA or RIA.

Such assays can be used to determine the dissociation constant of the antibody. The phrase “dissociation constant” refers to the affinity of an antibody for an antigen. Specificity of binding between an antibody and an antigen exists if the dissociation constant (K_{D}=1/K, where K is the affinity constant) of the antibody is <1 μM, preferably <100 nM, and most preferably <0.1 nM. Antibody molecules will typically have a K_{D} in the lower ranges. K_{D}=[Ab-Ag]/[Ab][Ag] where [Ab] is the concentration at equilibrium of the antibody, [Ag] is the concentration at equilibrium of the antigen and [Ab-Ag] is the concentration at equilibrium of the antibody-antigen complex. Typically, the binding interactions between antigen and antibody include reversible noncovalent associations such as electrostatic attraction, Van der Waals forces and hydrogen bonds. This method of defining binding specificity applies to single heavy and/or light chains, CDRs, fusion proteins or fragments of heavy and/or light chains, that are specific for mesothelin if they bind mesothelin alone or in combination.

The antibodies can be detected and/or quantified using any of a number of well recognized immunological binding assays (see, e.g., U.S. Pat. Nos. 4,366,241; 4,376,110; 4,517,288; and 4,837,168). For a review of the general immunoassays, see also METHODS IN CELL BIOLOGY, VOL. 37, Asai, ed. Academic Press, Inc. New York (1993); BASIC AND CLINICAL IMMUNOLOGY 7TH EDITION, Stites & Terr, eds. (1991). Immunological binding assays (or immunoassays) typically utilize a ligand (e.g., mesothelin) to specifically bind to and often immobilize an antibody. The antibodies employed in immunoassays of the present invention are discussed in greater detail supra.

Immunoassays also often utilize a labeling agent to specifically bind to and label the binding complex formed by the ligand and the antibody. The labeling agent may itself be one of the moieties comprising the antibody/analyte complex, i.e., the anti-mesothelin antibody. Alternatively, the labeling agent may be a third moiety, such as another antibody, that specifically binds to the antibody/mesothelin protein complex.

In one aspect, a competitive assay is contemplated wherein the labeling agent is a second anti-mesothelin antibody bearing a label. The two antibodies then compete for binding to the immobilized mesothelin. Alternatively, in a non-competitive format, the mesothelin antibody lacks a label, but a second antibody specific to antibodies of the species from which the anti-mesothelin antibody is derived, e.g., murine, and which binds the anti-mesothelin antibody, is labeled.

Other proteins capable of specifically binding immunoglobulin constant regions, such as Protein A or Protein G may also be used as the label agent. These proteins are normal constituents of the cell walls of streptococcal bacteria. They exhibit a strong non-immunogenic reactivity with immunoglobulin constant regions from a variety of species (see, generally Kronval, et al., J. Immunol. 111:1401-1406 (1973); and Akerstrom, et al., J. Immunol. 135:2589-2542 (1985)).

Throughout the assays, incubation and/or washing steps may be required after each combination of reagents. Incubation steps can vary from about 5 seconds to several hours, preferably from about 5 minutes to about 24 hours. However, the incubation time will depend upon the assay format, antibody, volume of solution, concentrations, and the like. Usually, the assays will be carried out at ambient temperature, although they can be conducted over a range of temperatures, such as 10° C. to 40° C.

While the details of the immunoassays of the present invention may vary with the particular format employed, the method of detecting anti-mesothelin antibodies in a sample containing the antibodies generally comprises the steps of contacting the sample with an antibody which specifically reacts, under immunologically reactive conditions, to the mesothelin/antibody complex.

### IV. Production of Immunoconjugates

The anti-mesothelin antibodies of the invention can be linked to effector molecules (EM) through the EM carboxyl terminus, the EM amino terminus, through an interior amino acid residue of the EM such as cysteine, or any combination thereof. Similarly, the EM can be linked directly to heavy, light, Fc (constant region) or framework regions of the antibody. Linkage can occur through the antibody's amino or carboxyl termini, or through an interior amino acid residue. Further, multiple EM molecules (e.g., any one of from 2-10) can be linked to the anti-mesothelin antibody and/or multiple antibodies (e.g., any one of from 2-5) can be linked to an EM. The antibodies used in a multivalent immunoconjugate composition of the present invention can be directed to the same or different mesothelin epitopes.

Immunoconjugates include, but are not limited to, molecules in which there is a covalent linkage of a therapeutic agent to an antibody. A therapeutic agent is an agent with a particular biological activity directed against a particular target molecule or a cell bearing a target molecule. One of skill in the art will appreciate that therapeutic agents may include various drugs such as vinblastine, daunoinycin and the like, cytotoxins such as native or modified Pseudomonas exotoxin or Diphtheria toxin, encapsulating agents, (e.g., liposomes) which themselves contain pharmacological compositions such as doxorubicin or other drugs, radioactive agents such as ¹²⁵I, ³²P, ¹⁴C, ³H and ³⁵S and other labels, target moieties and ligands.

The choice of a particular therapeutic agent depends on the particular target molecule or cell and the biological effect is desired to evoke. Thus, for example, the therapeutic agent may be a cytotoxin which is used to bring about the death of a particular target cell. Conversely, where it is merely desired to invoke a non-lethal biological response, the therapeutic agent may be conjugated to a non-lethal pharmacological agent or a liposome containing a non-lethal pharmacological agent.

With the therapeutic agents and antibodies herein provided, one of skill can readily construct a variety of clones containing functionally equivalent nucleic acids, such as nucleic acids which differ in sequence but which encode the same EM or antibody sequence. Thus, the present invention provides nucleic acids encoding antibodies and conjugates and fusion proteins thereof.

A. Recombinant Methods

The nucleic acid sequences of the present invention can be prepared by any suitable method including, for example, cloning of appropriate sequences or by direct chemical synthesis by methods such as the phosphotriester method of Narang, et al., Meth. Enzymol. 68:90-99 (1979); the phosphodiester method of Brown, et al., Meth. Enzymol. 68:109-151 (1979); the diethylphosphoramidite method of Beaucage, et al., Tetra. Lett. 22:1859-1862 (1981); the solid phase phosphoramidite triester method described by Beaucage & Caruthers, Tetra. Letts. 22(20):1859-1862 (1981), e.g., using an automated synthesizer as described in, for example, Needham-VanDevanter, et al. Nucl. Acids Res. 12:6159-6168 (1984); and, the solid support method of U.S. Pat. No. 4,458,066. Chemical synthesis produces a single stranded oligonucleotide. This may be converted into double stranded DNA by hybridization with a complementary sequence, or by polymerization with a DNA polymerase using the single strand as a template. One of skill would recognize that while chemical synthesis of DNA is limited to sequences of about 100 bases, longer sequences may be obtained by the ligation of shorter sequences.

In a preferred embodiment, the nucleic acid sequences of this invention are prepared by cloning techniques. Examples of appropriate cloning and sequencing techniques, and instructions sufficient to direct persons of skill through many cloning exercises are found in Sambrook, et al., MOLECULAR CLONING: A LABORATORY MANUAL (2ND ED.), Vols. 1-3, Cold Spring Harbor Laboratory (1989)), Berger and Kimmel (eds.), GUIDE TO MOLECULAR CLONING TECHNIQUES, Academic Press, Inc., San Diego Calif. (1987)), or Ausubel, et al. (eds.), CURRENT PROTOCOLS IN MOLECULAR BIOLOGY, Greene Publishing and Wiley-Interscience, NY (1987). Product information from manufacturers of biological reagents and experimental equipment also provide useful information. Such manufacturers include the SIGMA chemical company (Saint Louis, Mo.), R&D systems (Minneapolis, Minn.), Pharmacia LKB Biotechnology (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersberg, Md.), Fluka Chemica-Biochemika Analytika (Fluka Chemie AG, Buchs, Switzerland), Invitrogen, San Diego, Calif., and Applied Biosystems (Foster City, Calif.), as well as many other commercial sources known to one of skill.

Nucleic acids encoding native EM or anti-mesothelin antibodies can be modified to form the EM, antibodies, or immunoconjugates of the present invention. Modification by site-directed mutagenesis is well known in the art. Nucleic acids encoding EM or anti-mesothelin antibodies can be amplified by in vitro methods. Amplification methods include the polymerase chain reaction (PCR), the ligase chain reaction (LCR), the transcription-based amplification system (TAS), the self-sustained sequence replication system (3SR). A wide variety of cloning methods, host cells, and in vitro amplification methodologies are well known to persons of skill.

In a preferred embodiment, immunoconjugates are prepared by inserting the cDNA which encodes an anti-mesothelin scFv antibody into a vector which comprises the cDNA encoding the EM. The insertion is made so that the scFv and the EM are read in frame, that is in one continuous polypeptide which contains a functional Fv region and a functional EM region. In a particularly preferred embodiment, cDNA encoding a diphtheria toxin fragment is ligated to a scFv so that the toxin is located at the carboxyl terminus of the scFv. In a most preferred embodiment, cDNA encoding PE is ligated to a scFv so that the toxin is located at the amino terminus of the scFv.

Once the nucleic acids encoding an EM, anti-mesothelin antibody, or an immunoconjugate of the present invention are isolated and cloned, one may express the desired protein in a recombinantly engineered cell such as bacteria, plant, yeast, insect and mammalian cells. It is expected that those of skill in the art are knowledgeable in the numerous expression systems available for expression of proteins including E. coli, other bacterial hosts, yeast, and various higher eucaryotic cells such as the COS, CHO, HeLa and myeloma cell lines. No attempt to describe in detail the various methods known for the expression of proteins in prokaryotes or eukaryotes will be made. In brief, the expression of natural or synthetic nucleic acids encoding the isolated proteins of the invention will typically be achieved by operably linking the DNA or cDNA to a promoter (which is either constitutive or inducible), followed by incorporation into an expression cassette. The cassettes can be suitable for replication and integration in either prokaryotes or eukaryotes. Typical expression cassettes contain transcription and translation terminators, initiation sequences, and promoters useful for regulation of the expression of the DNA encoding the protein. To obtain high level expression of a cloned gene, it is desirable to construct expression cassettes which contain, at the minimum, a strong promoter to direct transcription, a ribosome binding site for translational initiation, and a transcription/translation terminator. For E. coli this includes a promoter such as the T7, trp, lac, or lambda promoters, a ribosome binding site and preferably a transcription termination signal. For eukaryotic cells, the control sequences can include a promoter and preferably an enhancer derived from immunoglobulin genes, SV40, cytomegalovirus, and a polyadenylation sequence, and may include splice donor and acceptor sequences. The cassettes of the invention can be transferred into the chosen host cell by well-known methods such as calcium chloride transformation or electroporation for E. coli and calcium phosphate treatment, electroporation or lipofection for mammalian cells. Cells transformed by the cassettes can be selected by resistance to antibiotics conferred by genes contained in the cassettes, such as the amp, gpt, neo and hyg genes.

One of skill would recognize that modifications can be made to a nucleic acid encoding a polypeptide of the present invention (i.e., anti-mesothelin antibody, PE, or an immunoconjugate formed from their combination) without diminishing its biological activity. Some modifications may be made to facilitate the cloning, expression, or incorporation of the targeting molecule into a fusion protein. Such modifications are well known to those of skill in the art and include, for example, termination codons, a methionine added at the amino terminus to provide an initiation, site, additional amino acids placed on either terminus to create conveniently located restriction sites, or additional amino acids (such as poly His) to aid in purification steps.

In addition to recombinant methods, the immunoconjugates, EM, and antibodies of the present invention can also be constructed in whole or in part using standard peptide synthesis. Solid phase synthesis of the polypeptides of the present invention of less than about 50 amino acids in length may be accomplished by attaching the C-terminal amino acid of the sequence to an insoluble support followed by sequential addition of the remaining amino acids in the sequence. Techniques for solid phase synthesis are described by Barany & Merrifield, THE PEPTIDES: ANALYSIS, SYNTHESIS, BIOLOGY. VOL. 2: SPECIAL METHODS IN PEPTIDE SYNTHESIS, PART A. pp. 3-284; Merrifield, et al. J. Am. Chem. Soc. 85:2149-2156 (1963), and Stewart, et al., SOLID PHASE PEPTIDE SYNTHESIS, 2ND ED., Pierce Chem. Co., Rockford, Ill. (1984). Proteins of greater length may be synthesized by condensation of the amino and carboxyl termini of shorter fragments. Methods of forming peptide bonds by activation of a carboxyl terminal end (e.g., by the use of the coupling reagent N,N′-dicycylohexylcarbodiimide) are known to those of skill.

B. Purification

Once expressed, the recombinant immunoconjugates, antibodies, and/or effector molecules of the present invention can be purified according to standard procedures of the art, including ammonium sulfate precipitation, affinity columns, column chromatography, and the like (see, generally, R. Scopes, PROTEIN PURIFICATION, Springer-Verlag, N.Y. (1982)). Substantially pure compositions of at least about 90 to 95% homogeneity are preferred, and 98 to 99% or more homogeneity are most preferred for pharmaceutical uses. Once purified, partially or to homogeneity as desired, if to be used therapeutically, the polypeptides should be substantially free of endotoxin.

Methods for expression of single chain antibodies and/or refolding to an appropriate active form, including single chain antibodies, from bacteria such as E. coli have been described and are well-known and are applicable to the antibodies of this invention. See, Buchner, et al., Anal. Biochem. 205:263-270 (1992); Pluckthun, Biotechnology 9:545 (1991); Huse, et al., Science 246:1275 (1989) and Ward, et al., Nature 341:544 (1989), all incorporated by reference herein.

Often, functional heterologous proteins from E. coli or other bacteria are isolated from inclusion bodies and require solubilization using strong denaturants, and subsequent refolding. During the solubilization step, as is well-known in the art, a reducing agent must be present to separate disulfide bonds. An exemplary buffer with a reducing agent is: 0.1 M Tris pH 8, 6 M guanidine, 2 mM EDTA, 0.3 M DTE (dithioerythritol). Reoxidation of the disulfide bonds can occur in the presence of low molecular weight thiol reagents in reduced and oxidized form, as described in Saxena, et al., Biochemistry 9: 5015-5021 (1970), incorporated by reference herein, and especially as described by Buchner, et al., supra.

Renaturation is typically accomplished by dilution (e.g., 100-fold) of the denatured and reduced protein into refolding buffer. An exemplary buffer is 0.1 M Tris, pH 8.0, 0.5 M L-arginine, 8 mM oxidized glutathione (GSSG), and 2 mM EDTA.

As a modification to the two chain antibody purification protocol, the heavy and light chain regions are separately solubilized and reduced and then combined in the refolding solution. A preferred yield is obtained when these two proteins are mixed in a molar ratio such that a 5 fold molar excess of one protein over the other is not exceeded. It is desirable to add excess oxidized glutathione or other oxidizing low molecular weight compounds to the refolding solution after the redox-shuffling is completed.

### V. Therapeutic Moieties and Detectable Labels

In some embodiments, the antibodies of the invention can be coupled to therapeutic moieties or to detectable labels. When the therapeutic moiety is a cytotoxin, the resulting chimeric molecule is referred to as an immunotoxin. Exemplary toxins include ricin, abrin, diphtheria toxin and subunits thereof, as well as botulinum toxins A through F. These toxins are readily available from commercial sources (e.g., Sigma Chemical Company, St. Louis, Mo.). Diphtheria toxin is isolated from Corynebacterium diphtheriae. Ricin is the lectin RCA60 from Ricinus communis (Castor bean). The term also references toxic variants thereof. For example, see, U.S. Pat. Nos. 5,079,163 and 4,689,401. Ricinus communis agglutinin (RCA) occurs in two forms designated RCA₆₀ and RCA₁₂₀ according to their molecular weights of approximately 65 and 120 kD, respectively (Nicholson & Blaustein, J. Biochim. Biophys. Acta 266:543 (1972)). The A chain is responsible for inactivating protein synthesis and killing cells. The B chain binds ricin to cell-surface galactose residues and facilitates transport of the A chain into the cytosol (Olsnes, et al., Nature 249:627-631 (1974) and U.S. Pat. No. 3,060,165).

Abrin includes toxic lectins from Abrus precatorius. The toxic principles, abrin a, b, c, and d, have a molecular weight of from about 63 and 67 kD and are composed of two disulfide-linked polypeptide chains A and B. The A chain inhibits protein synthesis; the B-chain (abrin-b) binds to D-galactose residues (see, Funatsu, et al., Agr. Biol. Chem. 52:1095 (1988); and Olsnes, Methods Enzymol. 50:330-335 (1978)).

In preferred embodiments of the present invention, the toxin is Pseudomonas exotoxin (“PE”). The term “Pseudomonas exotoxin” as used herein refers to a PE that has been modified from the native sequence to reduce or to eliminate non-specific binding. Such modifications may include, but are not limited to, elimination of domain Ia, various amino acid deletions in domains Ib, II and III, single amino acid substitutions and the addition of one or more sequences at the carboxyl terminus such as KDEL and REDL. See Siegall, et al., J. Biol. Chem. 264:14256-14261 (1989). In a preferred embodiment, the cytotoxic fragment of PE retains at least 50%, preferably 75%, more preferably at least 90%, and most preferably 95% of the cytotoxicity of native PE when delivered to a cell bearing mesothelin. In a most preferred embodiment, the cytotoxic fragment is more toxic than native PE.

Native Pseudomonas exotoxin A (“PE”) is an extremely active monomeric protein (molecular weight 66 kD), secreted by Pseudomonas aeruginosa, which inhibits protein synthesis in eukaryotic cells. The native 613 amino acid sequence of PE is provided in U.S. Pat. No. 5,602,095, incorporated herein by reference. The method of action is inactivation of the ADP-ribosylation of elongation factor 2 (EF-2). The exotoxin contains three structural domains that act in concert to cause cytotoxicity. Domain Ia (amino acids 1-252) mediates cell binding. Domain II (amino acids 253-364) is responsible for translocation into the cytosol and domain III (amino acids 400-613) mediates ADP ribosylation of elongation factor 2. The function of domain Ib (amino acids 365-399) remains undefined, although a large part of it, amino acids 365-380, can be deleted without loss of cytotoxicity. See Siegall, et al., (1989), supra.

The term “PE” as used herein includes cytotoxic fragments of the native sequence, and conservatively modified variants of native PE and its cytotoxic fragments. Cytotoxic fragments of PE include those which are cytotoxic with or without subsequent proteolytic or other processing in the target cell (e.g., as a protein or pre-protein). Cytotoxic fragments and variants of PE have been investigated for years as agents for clinical use; several of these fragments and variants are described below. For convenience, residues of PE which are deleted or mutated are typically referred to in the art by their position in the 613 amino acid sequence of native PE. As noted, the 613-amino acid sequence of native PE is well known in the art.

In preferred embodiments, the PE has been modified to reduce or eliminate non-specific cell binding. Frequently, this is achieved by deleting domain Ia. as taught in U.S. Pat. No. 4,892,827, although it can also be achieved by, for example, mutating certain residues of domain Ia. U.S. Pat. No. 5,512,658, for instance, discloses that a mutated PE in which Domain Ia is present but in which the basic residues of domain Ia at positions 57, 246, 247, and 249 are replaced with acidic residues (glutamic acid, or “E”)) exhibits greatly diminished non-specific cytotoxicity. This mutant form of PE is sometimes referred to as “PE4E”.

One derivative of PE in which Domain Ia is deleted has a molecular weight of 40 KDa and is correspondingly known as PE40. See, Pai, et al., Proc. Nat'l Acad. Sci. USA 88:3358-62 (1991); and Kondo, et al., J Biol Chem. 263:9470-9475 (1988). PE35 is a 35 kD carboxyl-terminal fragment of PE in which amino acid residues 1-279 have deleted and the molecule commences with a methionine residue at position 280, followed by amino acids 281-364 and 381-613 of native PE. PE35 and PE40 are disclosed, for example, in U.S. Pat. Nos. 5,602,095 and 4,892,827.

In some preferred embodiments, the cytotoxic fragment PE38 is employed. PE38 is a truncated PE pro-protein composed of PE amino acids 253-364 and 381-613 which is activated to its cytotoxic form upon processing within a cell (see e.g., U.S. Pat. No. 5,608,039, and Pastan et al., Biochim. Biophys. Acta 1333:C1-C6 (1997)). In some embodiments, the lysine residues at positions 590 and 606 of PE in PE38 are mutated to glutamines, while the lysine at position 613 is mutated to arginine, to create a form known as “PE38QQR.” See, e.g., Debinski and Pastan, Bioconj. Chem., 5: 40-46 (1994). This form of PE was originally developed in the course of increasing the homogeneity of immunotoxins formed by chemically coupling the PE molecules to the targeting antibodies.

As noted above, some or all of domain 1b may be deleted, and the remaining portions joined by a linker or directly by a peptide bond. Some of the amino portion of domain II may be deleted. And, the C-terminal end may contain the native sequence of residues 609-613 (REDLK), or may contain a variation found to maintain the ability of the construct to translocate into the cytosol, such as REDL or KDEL, and repeats of these sequences. See, e.g., U.S. Pat. Nos. 5,854,044; 5,821,238; and 5,602,095 and WO 99/51643. While in preferred embodiments, the PE is PE4E, PE40, PE38, or PE38QQR, any form of PE in which non-specific cytotoxicity has been eliminated or reduced to levels in which significant toxicity to non-targeted cells does not occur can be used in the immunotoxins of the present invention so long as it remains capable of translocation and EF-2 ribosylation in a targeted cell.

In some preferred embodiments, the toxicity of the PE is increased by mutating the arginine (R) at position 490 of the native sequence of PE. The R is mutated to an amino acid having an aliphatic side chain that does not comprise a hydroxyl. Thus, the R can be mutated to glycine (G), alanine (A), valine (V), leucine (L), or isoleucine (I). In preferred embodiments, the substituent is G, A, or I. Alanine is the most preferred. Surprisingly, the mutation of the arginine at position 490 to alanine doubles the toxicity of the PE molecule. The discovery of this method of increasing the toxicity of PE is disclosed in co-owned international application PCT/US2004/039617, which is incorporated herein by reference.

A. Conservatively Modified Variants of PE

Conservatively modified variants of PE or cytotoxic fragments thereof have at least 80% sequence similarity, preferably at least 85% sequence similarity, more preferably at least 90% sequence similarity, and most preferably at least 95% sequence similarity at the amino acid level, with the PE of interest, such as PE38.

The term “conservatively modified variants” applies to both amino acid and nucleic acid sequences. With respect to particular nucleic acid sequences, conservatively modified variants refer to those nucleic acid sequences which encode identical or essentially identical amino acid sequences, or if the nucleic acid does not encode an amino acid sequence, to essentially identical nucleic acid sequences. Because of the degeneracy of the genetic code, a large number of functionally identical nucleic acids encode any given polypeptide. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid.

B. Assaying for Cytotoxicity of PE

Pseudomonas exotoxins employed in the invention can be assayed for the desired level of cytotoxicity by assays well known to those of skill in the art. Exemplary toxicity assays are described in, e.g., WO 00/73346, Example 2. Thus, cytotoxic fragments of PE and conservatively modified variants of such fragments can be readily assayed for cytotoxicity. A large number of candidate PE molecules can be assayed simultaneously for cytotoxicity by methods well known in the art. For example, subgroups of the candidate molecules can be assayed for cytotoxicity. Positively reacting subgroups of the candidate molecules can be continually subdivided and reassayed until the desired cytotoxic fragment(s) is identified. Such methods allow rapid screening of large numbers of cytotoxic fragments or conservative

C. Other Therapeutic Moieties

Antibodies of the present invention can also be used to target any number of different diagnostic or therapeutic compounds to cells expressing mesothelin on their surface. Thus, an antibody of the present invention, such as an anti-mesothelin scFv, may be attached directly or via a linker to a drug that is to be delivered directly to cells bearing mesothelin. Therapeutic agents include such compounds as nucleic acids, proteins, peptides, amino acids or derivatives, glycoproteins, radioisotopes, lipids, carbohydrates, or recombinant viruses. Nucleic acid therapeutic and diagnostic moieties include antisense nucleic acids, derivatized oligonucleotides for covalent cross-linking with single or duplex DNA, and triplex forming oligonucleotides.

Alternatively, the molecule linked to an anti-mesothelin antibody may be an encapsulation system, such as a liposome or micelle that contains a therapeutic composition such as a drug, a nucleic acid (e.g. an antisense nucleic acid), or another therapeutic moiety that is preferably shielded from direct exposure to the circulatory system. Means of preparing liposomes attached to antibodies are well known to those of skill in the art. See, for example, U.S. Pat. No. 4,957,735; and Connor, et al., Pharm. Ther. 28:341-365 (1985).

D. Detectable Labels

Antibodies of the present invention may optionally be covalently or non-covalently linked to a detectable label. Detectable labels suitable for such use include any composition detectable by spectroscopic, photochemical, biochemical, immunochemical, electrical, optical or chemical means. Useful labels in the present invention include magnetic beads (e.g. DYNABEADS), fluorescent dyes (e.g., fluorescein isothiocyanate, Texas red, rhodamine, green fluorescent protein, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and calorimetric labels such as colloidal gold or colored glass or plastic (e.g. polystyrene, polypropylene, latex) beads.

Means of detecting such labels are well known to those of skill in the art. Thus, for example, radiolabels may be detected using photographic film or scintillation counters, fluorescent markers may be detected using a photodetector to detect emitted illumination. Enzymatic labels are typically detected by providing the enzyme with a substrate and detecting the reaction product produced by the action of the enzyme on the substrate, and calorimetric labels are detected by simply visualizing the colored label.

E. Conjugation to the Antibody

In a non-recombinant embodiment of the invention, effector molecules, e.g., therapeutic, diagnostic, or detection moieties, are linked to the anti-mesothelin antibodies of the present invention using any number of means known to those of skill in the art. Both covalent and noncovalent attachment means may be used with anti-mesothelin antibodies of the present invention.

The procedure for attaching an effector molecule to an antibody will vary according to the chemical structure of the EM. Polypeptides typically contain a variety of functional groups; e.g., carboxylic acid (COOH), free amine (—NH₂) or sulfhydryl (—SH) groups, which are available for reaction with a suitable functional group on an antibody to result in the binding of the effector molecule.

Alternatively, the antibody is derivatized to expose or to attach additional reactive functional groups. The derivatization may involve attachment of any of a number of linker molecules, such as those available from Pierce Chemical Company (Rockford Ill.).

A “linker”, as used herein, is a molecule that is used to join the antibody to the effector molecule. The linker is capable of forming covalent bonds to both the antibody and to the effector molecule. Suitable linkers are well known to those of skill in the art and include, but are not limited to, straight or branched-chain carbon linkers, heterocyclic carbon linkers, or peptide linkers. Where the antibody and the effector molecule are polypeptides, the linkers may be joined to the constituent amino acids through their side groups (e.g., through a disulfide linkage to cysteine). However, in a preferred embodiment, the linkers will be joined to the alpha carbon amino and carboxyl groups of the terminal amino acids.

In some circumstances, it is desirable to free the effector molecule from the antibody when the immunoconjugate has reached its target site. Therefore, in these circumstances, immunoconjugates will comprise linkages which are cleavable in the vicinity of the target site. Cleavage of the linker to release the effector molecule from the antibody may be prompted by enzymatic activity or conditions to which the immunoconjugate is subjected either inside the target cell or in the vicinity of the target site. When the target site is a tumor, a linker which is cleavable under conditions present at the tumor site (e.g. when exposed to tumor-associated enzymes or acidic pH) may be used.

In view of the large number of methods that have been reported for attaching a variety of radiodiagnostic compounds, radiotherapeutic compounds, drugs, toxins, and other agents to antibodies one skilled in the art will be able to determine a suitable method for attaching a given agent to an antibody or other polypeptide.

### VI. Pharmaceutical Compositions and Administration

The antibody and/or immunoconjugate compositions of this invention (i.e., PE linked to an MN or MB antibody), are particularly useful for parenteral administration, such as intravenous administration or administration into a body cavity or lumen of an organ. For example, ovarian malignancies may be treated by intravenous administration or by localized delivery to the tissue surrounding the tumor. To treat mesotheliomas, pharmaceutical compositions of this invention comprising anti-mesothelin antibodies can be administered directly into the pleural or peritoneal cavities.

The compositions for administration will commonly comprise a solution of the antibody and/or immunoconjugate dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of fusion protein in these formulations can vary widely, and will be selected primarily based on fluid volumes, viscosities, body weight and the like in accordance with the particular mode of administration selected and the patient's needs.

Thus, a typical pharmaceutical immunotoxin composition of the present invention for intravenous administration would be about 0.1 to 10 mg per patient per day. Dosages from 0.1 up to about 100 mg per patient per day may be used, particularly if the drug is administered to a secluded site and not into the circulatory or lymph system, such as into a body cavity or into a lumen of an organ. Actual methods for preparing administrable compositions will be known or apparent to those skilled in the art and are described in more detail in such publications as REMINGTON'S PHARMACEUTICAL SCIENCE, 19TH ED., Mack Publishing Company, Easton, Pa. (1995). As noted in the Background, clinical trials of the anti-mesothelin immunotoxin SS1P are underway, and dosage information from those trials can also be used to guide administration of immunotoxins using antibodies of the present invention.

The compositions of the present invention can be administered for therapeutic treatments. In therapeutic applications, compositions are administered to a patient suffering from a disease, in an amount sufficient to cure or at least partially arrest the disease and its complications. An amount adequate to accomplish this is defined as a “therapeutically effective dose.” Amounts effective for this use will depend upon the severity of the disease and the general state of the patient's health. An effective amount of the compound is that which provides either subjective relief of a symptom(s) or an objectively identifiable improvement as noted by the clinician or other qualified observer.

Single or multiple administrations of the compositions are administered depending on the dosage and frequency as required and tolerated by the patient. In any event, the composition should provide a sufficient quantity of the proteins of this invention to effectively treat the patient. Preferably, the dosage is administered once but may be applied periodically until either a therapeutic result is achieved or until side effects warrant discontinuation of therapy. Generally, the dose is sufficient to treat or ameliorate symptoms or signs of disease without producing unacceptable toxicity to the patient.

Controlled release parenteral formulations of the immunoconjugate compositions of the present invention can be made as implants, oily injections, or as particulate systems. For a broad overview of protein delivery systems see, Banga, A. J., THERAPEUTIC PEPTIDES AND PROTEINS: FORMULATION, PROCESSING, AND DELIVERY SYSTEMS, Technomic Publishing Company, Inc., Lancaster, Pa., (1995) incorporated herein by reference. Particulate systems include microspheres, microparticles, microcapsules, nanocapsules, nanospheres, and nanoparticles. Microcapsules contain the therapeutic protein as a central core. In microspheres the therapeutic is dispersed throughout the particle. Particles, microspheres, and microcapsules smaller than about 1 μm are generally referred to as nanoparticles, nanospheres, and nanocapsules, respectively. Capillaries have a diameter of approximately 5 μm so that only nanoparticles are administered intravenously. Microparticles are typically around 100 μm in diameter and are administered subcutaneously or intramuscularly. See, e.g., Kreuter, J., COLLOIDAL DRUG DELIVERY SYSTEMS, J. Kreuter, ed., Marcel Dekker, Inc., New York, N.Y., pp. 219-342 (1994); and Tice & Tabibi, TREATISE ON CONTROLLED DRUG DELIVERY, A. Kydonieus, ed., Marcel Dekker, Inc. New York, N.Y., pp. 315-339, (1992) both of which are incorporated herein by reference.

Polymers can be used for ion-controlled release of immunoconjugate compositions of the present invention. Various degradable and nondegradable polymeric matrices for use in controlled drug delivery are known in the art (Langer, R., Accounts Chem. Res. 26:537-542 (1993)). For example, the block copolymer, polaxamer 407 exists as a viscous yet mobile liquid at low temperatures but forms a semisolid gel at body temperature. It has shown to be an effective vehicle for formulation and sustained delivery of recombinant interleukin-2 and urease (Johnston, et al., Pharm. Res. 9:425-434 (1992); and Pec, et al., J. Parent. Sci. Tech. 44(2):58-65 (1990)). Alternatively, hydroxyapatite has been used as a microcarrier for controlled release of proteins (Ijntema, et al., Int. J. Pharm. 112:215-224 (1994)). In yet another aspect, liposomes are used for controlled release as well as drug targeting of the lipid-capsulated drug (Betageri, et al., LIPOSOME DRUG DELIVERY SYSTEMS, Technomic Publishing Co., Inc., Lancaster, Pa. (1993)). Numerous additional systems for controlled delivery of therapeutic proteins are known. See, e.g., U.S. Pat. Nos. 5,055,303, 5,188,837, 4,235,871, 4,501,728, 4,837,028 4,957,735 and 5,019,369, 5,055,303; 5,514,670; 5,413,797; 5,268,164; 5,004,697; 4,902,505; 5,506,206, 5,271,961; 5,254,342 and 5,534,496, each of which is incorporated herein by reference.

Among various uses of the immunotoxins of the present invention are included a variety of disease conditions caused by specific human cells that may be eliminated by the toxic action of the fusion protein. One preferred application for the immunotoxins of the invention is the treatment of malignant cells expressing mesothelin. Exemplary malignant cells include ovarian, stomach and squamous cell cancers as well as mesotheliomas.

### VII. Kits and In Vitro Uses

In another embodiment, this invention provides for kits for the detection of mesothelin or an immunoreactive fragment thereof, (i.e., collectively, a “mesothelin protein”) in a biological sample. A “biological sample” as used herein is a sample of biological tissue or fluid that contains mesothelin. Such samples include, but are not limited to, tissue from biopsy, sputum, amniotic fluid, blood, and blood cells (e.g., white cells). Biological samples also include sections of tissues, such as frozen sections taken for histological purposes. A biological sample is typically obtained from a multicellular eukaryote, preferably a mammal such as rat, mouse, cow, dog, guinea pig, or rabbit, more preferably from a primate, such as a macaque, chimpanzee, and most preferably from a human.

Kits will typically comprise an anti-mesothelin antibody of the present invention. In some embodiments, the anti-mesothelin antibody will be an anti-mesothelin Fv fragment, such as a scFv fragment.

In addition the kits will typically include instructional materials disclosing means of use of an antibody of the present invention (e.g. for detection of mesothelial cells in a sample). The kits may also include additional components to facilitate the particular application for which the kit is designed. Thus, for example, the kit may additionally contain means of detecting the label (e.g. enzyme substrates for enzymatic labels, filter sets to detect fluorescent labels, appropriate secondary labels such as a sheep anti-mouse-HRP, or the like). The kits may additionally include buffers and other reagents routinely used for the practice of a particular method. Such kits and appropriate contents are well known to those of skill in the art.

In one embodiment of the present invention, the diagnostic kit comprises an immunoassay. As described above, although the details of the immunoassays of the present invention may vary with the particular format employed, the method of detecting mesothelin in a biological sample generally comprises the steps of contacting the biological sample with an antibody which specifically reacts, under immunologically reactive conditions, to mesothelin. The antibody is allowed to bind to mesothelin under immunologically reactive conditions, and the presence of the bound antibody is detected directly or indirectly. The anti-mesothelin antibody may be used, for example, as the capture antibody of an ELISA, or as a second antibody to bind to mesothelin captured by the capture antibody. As is known in the art, the presence of the second antibody is typically then detected.

Due to the increased affinity of antibodies developed by the methods taught herein, and of the antibodies designated MN and MB in particular, the antibodies provided herein are especially useful as diagnostic agents and in in vitro assays to detect the presence of mesothelin in biological samples. For example, the antibodies MN and MB and variants of these antibodies as described herein can be used as the targeting moieties of immunoconjugates in immunohistochemical assays to determine whether a sample contains cells expressing mesothelin. If the sample is one taken from a tissue of a patient which should not normally express mesothelin, detection of mesothelin would indicate either that the patient has a cancer characterized by the presence of mesothelin-expressing cells, or that a treatment for such a cancer has not yet been successful at eradicating the cancer.

In another set of uses for the invention, immunotoxins targeted by antibodies of the invention can be used to purge targeted cells from a population of cells in a culture. Thus, for example, cells cultured from a patient having a cancer expressing mesothelin can be purged of cancer cells by contacting the culture with immunotoxins which use an MB antibody or preferably an MN antibody (such as scFvs) as a targeting moiety.

Although the present invention has been described in some detail by way of illustration and example for purposes of clarity of understanding, it will be obvious that certain changes and modifications may be practiced within the scope of the appended claims.

### EXAMPLES

### Example 1

This Example sets forth the materials and methods used in the studies underlying the present invention.

Generation of a Mesothelin-Fc Fusion Protein by Mammalian Cells. The extra-cellular domain of the human mesothelin was expressed as a fusion protein with rabbit IgG Fc in HEK 293T cells. The DNA fragment encoding rabbit IgG Fc (“RFc”) was amplified by PCR using the plasmid pγB1-12,14 for the RFc (amino acids 96-323 SEQ ID NO:9), Swissprot, kindly provided by Dr. Rose G. Mage, NIH, [Bernstein, K. E. et al., Immunogenetics 18:387-97 (1983)]) as the template and inserted between Sfi I and Sac II sites of pSecTag2 (Invitrogen, Carlsbad, Calif.). cDNA for the extracellular domain of mesothelin was inserted between the Sac II and Not I to obtain the plasmid pOND-rFc-Meso. Primers used were as follows: Meso Forward; 5′-AGA TAG AGT CCG CGG GGA GGT GAA GTG GAG AAG ACA GCC TGT-3′ (SEQ ID NO:10), Meso Reverse; 5′-TTG TAT AGC GGC CGC TCA TCC CCC CGA GAG GGC CTC TTG CAC-3′ (SEQ ID NO:11). The plasmid was transfected into 293T cells by Lipofectamine reagent (Invitrogen). The mesothelin-Fc protein harvested from the culture supernatant and purified with Hi-trap protein A column (Amersham Biosciences Corp., Piscataway, N.J.). The purified proteins were quantitated by Coomasie Blue (Pierce, Rockford, Ill.) and checked on SDS-PAGE gel.

Generation of a Mesothelin Protein by E. Coli. The mesothelin gene was cloned from the IMAGE cDNA cone ID 5209096 using PCR and put it into pMAL-p2X (NEB, Beverly, Mass.) which has a tobacco etch virus (TEV) cleavage site. The resulting plasmid pMH103 encodes a fusion protein consisting of the malE signal sequence, malE and mesothelin. The malE gene encodes the maltose binding protein (MBP). The fusion protein is directed to the periplasm of E. coli. The secreted proteins were separated with recombinant TEV (Invitrogen). The samples were applied to an amylase column for removal of MBP. The purity of bacterial mesothelin was over 95%.

Cells. The pancreatic cancer cell line Panc 3.014 was obtained from Dr. Elizabeth Jaffee (Johns Hopkins Medical Institute, Baltimore, Md.) (Thomas, A. M. et al., J Exp Med 200:297-306 (2004)). It was maintained in RPMI-1640 Medium supplemented with 20% fetal bovine serum (FBS), 200 μM L-glutamine, 50 units/ml Penicillin, 100 μg/ml Streptomycin, 1% non-essential amino acids (NEAA), 1% sodium pyruvate, and 2 units/ml human insulin. A human squamous cell carcinoma of mesothelioma cells, NCI-H226, was obtained from Dr. Isaiah J. Fidler (The University of Texas MD Anderson Cancer Center, Houston, Tex.). The cells were maintained in modified Eagle's medium (MEM) supplemented with 10% FBS, 2 mM L-glutamine, 1 mM sodium pyruvate and 1% NEAA. The human ovarian carcinoma cell line A1847 and human cervical carcinoma cell line Hela were grown in Dulbecco's modified Eagle's medium (DMEM) supplemented with 10% FBS, 100 IU/ml penicillin, and 100 μg/ml streptomycin.

Immunization. Female mesothelin-deficient mice (6-8 weeks old) were immunized four times with the pcDNA3CAK1-9 plasmid intradermally (50 μg in 50 μl of phosphate buffered saline [“PBS”]) with 2-week intervals, and one boost was given with the mesothelin-Fc intraperitoneally (50 μg in 100 μl of PBS) before fusion (Bera, T. K. et al., Mol Cell Biol 20:2902-6 (2000)). Spleens were harvested 84-90 hours after the last boost for cell fusion. All animals were maintained in accordance with institutional guidelines.

Cell Fusion. Cell fusions of the splenocytes and the SP2/0 cells were carried out according to a standard fusion protocol (Nelson, P. N. et al., Mol Pathol 53:111-7 (2000)). Fourteen days after fusion, the supernatants were harvested and screened for antibody production by ELISA. The selected hybridomas were grown in a CELLINE flask (INTEGRA Biosciences, Chur, Switzerland) and purified on a protein A column (Amersham Biosciences Corp.).

Screening by ELISA on Mesothelin-Fc Fusion Protein. Screening of hybridomas was performed by ELISA. Briefly, Nunc-Immuno plates (Nalge Nunc International, Rochester, N.Y.) were coated with 2 μg/ml of mesothelin-Fc protein. Plates were incubated overnight at 4° C. Then plates were blocked in blocking buffer (PBS with 25% DMEM, 5% FCS, 25 mM HEPES, 0.5% bovine serum albumin (“BSA”), and 0.1% Azide) for 30 minutes at room temperature, washed twice with washing buffer (PBS with 0.05% Tween 20), and incubated with 100 μl supernatant of hybridoma for 1 hour at room temperature. After washing, plates were incubated with a horseradish peroxidase (HRP)-conjugated goat anti-mouse antibody diluted 1:2000 in blocking buffer. Finally, plates were washed with washing buffer and 100 μl immunopure TMB substrate solution (Pierce) was added to each well. The color was allowed to develop for 2-5 minutes at room temperature and the reaction stopped by the addition of 50 μl 12N solution of sulfuric acid. The plates were read at OD 450 nm using an automated plate reader (Molecular Devices Corp., Sunnyvale, Calif.).

Antibody Quantification. For the determination of MAb concentration, Sandwich ELISA was used. Nunc-Immuno plates (Nalge Nunc International) were coated with 2 μg/ml of goat-anti-mouse IgG (Jackson ImmunoResearch Laboratories Inc., Grove, Pa.) in PBS overnight 4° C. One hundred μl of blocking buffer to each well was added and then incubated for 30 minutes. After washing, serial diluted samples were added. As a standard Ig, mouse immunoglobulin classes and subclasses (Zymed Laboratories Inc., San Francisco, Calif.) were added. After washing, the bound MAbs were detected by a I-hour incubation with HRP-labeled goat anti-mouse IgG (Jackson ImmunoResearch Laboratories) followed by tetramethylbenzidine (“TMB”) substrate kit (Pierce). After adding 2N—H₂SO₄ 50 μl, the plates were read at OD450. Standard curve was fit with the 4 parameter logistic curve fitting method.

Flow Cytometry. Cultured cells (2×10⁵) were dissociated with dissociation buffer (Sigma-Aldrich, St. Louis, Mo.). Each sample was washed twice in FACS buffer (PBS plus 5% FBS, and 0.1% Sodium azide). One hundred μl of each hybridoma supernatant was added to the cells and incubated for 1 hour at 4° C. Cells were then washed twice with FACS buffer, resuspended in 100 μl of a secondary antibody (R-PE conjugated goat anti-mouse IgG diluted 1:100; Biosource, Camarillo, Calif.) and incubated for another hour at 4° C. Finally, cells were washed twice and analyzed on a FACS Calibur machine (Becton-Dickinson, Franklin Drive, N.J.) using the Cell Quest software.

Isotyping. The isotype of selected MAbs was determined using a mouse immunoglobulin isotyping kit (Roche Applied Science, Indianapolis, Ind.).

Western Blot. Reactivity of anti-mesothelin MAbs to sodium dodecyl sulfate (SDS)-denatured antigen was tested in a Western blot analysis. Mesothelin-Fc and CD25-Fc were separated on 4-20% SDS polyacrylamide gels (Biorad, Hercules, Calif.) under reducing conditions. Proteins were transferred to a 0.2 μm Inmun-blot polyvinylidene difluoride membrane (BioRad) in transfer buffer (25 mM Tris-HCl, 192 mM glycine, 30% (v/v) methanol, pH 8.3) at 4° C. for 1 hour at 240 mA. After blocking with blocking solution (Roche Applied Science), the membrane was incubated with 1 μg/ml of each MAb for 1 hour at room temperature. The bound MAbs were detected with alkaline phosphatase-labeled goat anti-mouse IgG (Biosource) and BDIP/NBT substrate (Pierce).

Immunohistochemistry. Archival paraffin-embedded formaldehyde-fixed tissue sections from patients with mesothelioma were evaluated for mesothelin expression (Hassan, R. et al., Proc Am Soc Clin Oncol 21:29a (2002)). The sections were deparaffinized in xylene, followed by graded ethanol hydration into water. The sections were treated for antigen-retrieval either using: 1) a standard antigen unmasking solution (Vector Laboratories, Burlingame, Calif.) in a boiling water bath at 97° C. for 60 minutes, or 2) treatment with 3M urea in a boiling water bath for 60 minutes. These treatments were followed by blocking with 1% BSA in PBS for 30 minutes at 23° C., then incubation with primary antibodies in 1% BSA-PBS for 60 minutes at room temperature. Primary mouse antibodies 5B2 (Novocastra Laboratories Ltd, Newcastle upon Tyne, United Kingdom), MAb K1, MAb MB or MAb MN were used at (or at a preparation dilution equivalent to) 10 μg/ml. After washing in PBS, the sections were incubated with affinity-purified goat anti-mouse IgG conjugated to HRP (Jackson ImmunoResearch) at 25 μg/ml in 1% BSA-PBS for 30 min., followed by detection of peroxidase with diaminobenzidine-peroxide substrate solution for 10 minutes at 23° C. The sections were counterstained with hematoxylin.

### Example 2

This Example sets forth the results of studies underlying the present invention.

Generation of a Recombinant Mesothelin-Fc Fusion Protein and Generation of MAbs. To obtain MAbs that recognize the extracellular domain of mesothelin, we used a DNA immunization protocol followed by protein immunization for the final boost prior to cell fusion (Chowdhury, P. S. et al., J Immunol Methods 231:83-91 (1999)). The mesothelin-Fc protein used for immunization and screening was prepared from the medium of transfected HEK293T cells by purification on protein A-Sepharose (Nagata, S. et al., Clin Cancer Res 8:2345-55 (2002)). Six mesothelin deficient mice were injected intradermally with DNA prepared from the plasmid, pcDNA3CAK1-9. Blood was collected from the mice after multiple injections and the antibody titer was determined by ELISA on plates coated with mesothelin-Fc protein. The 2 mice with the highest titer (more than 10⁵) were sacrificed. Spleen cells from each mouse were fused to myeloma cells following standard procedures (Nelson, P. N. et al., Mol Pathol 53:111-7 (2000)). Supernatants from clones of hybridoma cells were screened using ELISA. To determine the specificity of the antibodies, the screening was performed on plates coated with mesothelin-Fc and CD25-Fc. The latter is a negative control that excludes antibodies reacting with the Fc portion of the fusion.

Seventeen hybridomas were identified that reacted selectively with mesothelin-Fc and not with CD25-Fc. The topographical epitopes of these MAbs were identified based on the mutual competition of all possible pairs of the MAbs (Nagata, S. et al., J Immunol Methods 292:141-55 (2004)). Two topographical epitope groups were identified. One group had 2 clones and contained MAb MB, while the other group had 15 clones and contained MAb MN, which competes with and belongs to the same epitope group as MAb K1.

The characteristics of the 2 new MAbs and the 2 standard anti-mesothelin MAbs, K1 and 5B2, are shown in Table 1. The affinities of the new anti-mesothelin MAbs as determined by Biacore (Kds of 1.0 and 0.6 nM) are much higher than previous MAbs. Because the new MAbs have high affinities for native mesothelin in the Biacore format, we examined their performance in other types of assays. In these studies we compared them with the 2 commercially available antibodies and found them to be useful in all the assays for mesothelin whereas the 2 others did not detect mesothelin in all assay conditions. The data is shown below and summarized in Table 1.

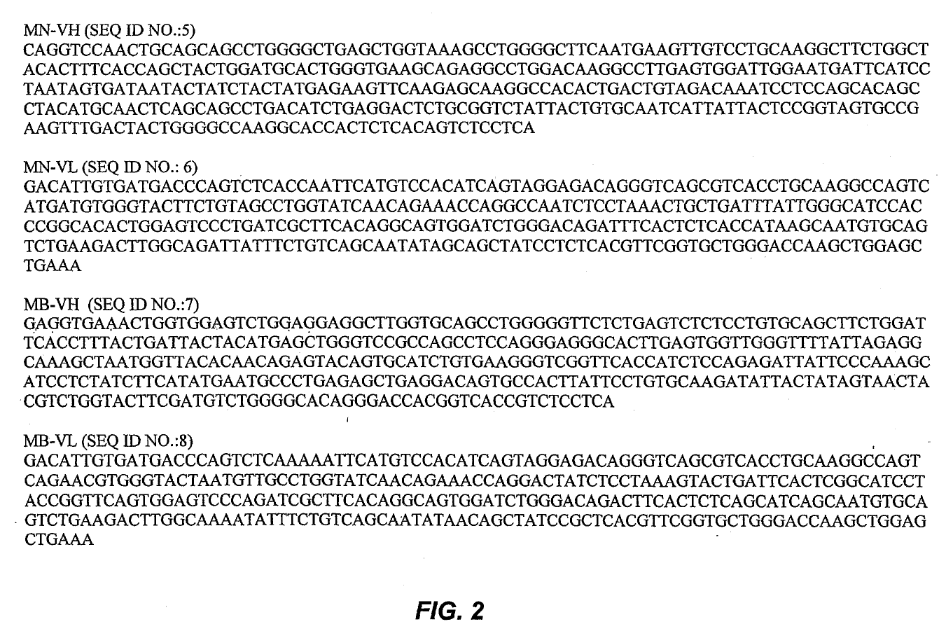

Flow Cytometry Analysis of the Anti-mesothelin Supernatants. The 17 supernatants were tested by FACS for their ability to bind to the Panc 3.014 mesothelin-expressing cell line. All 17 antibodies bound to Panc 3.014 cells but none of them bound to the A431 mesothelin-negative cells. One antibody from each epitope group, MN and MB, was selected for further study based on the highest signal on FACS. Representative FACS analysis of the new antibodies MN and MB, as well as MAb K1 and MAb 5B2 on H226 lung cancer cells, A1847 ovarian cancer cells, Hela cervical cancer cells and Panc 3.014 pancreatic cancer cells are shown in FIG. 2. The data in FIG. 2 show that MAbs MN and MB generated a large increase in fluorescence intensity compared to the cells incubated with the control anti-CD30 antibody. The signal was highest on H226 and Panc 3.014 cells followed by A1847 and Hela cells. MAb K1 also reacted with H226, Panc 3.014 and A1847 cells but the signal was weaker than with MAbs MN or MB. Using MAb 5B2, the fluorescence signal was weak on H226 and negative on A1847, Hela and Panc 3.014 cells. None of the antibodies showed detectable reactivity in mesothelin negative cells, A431. These data show that MAbs MN and MB are superior to the other MAbs for FACS analysis. The isotype of MAbs MN and MB are IgG2a.

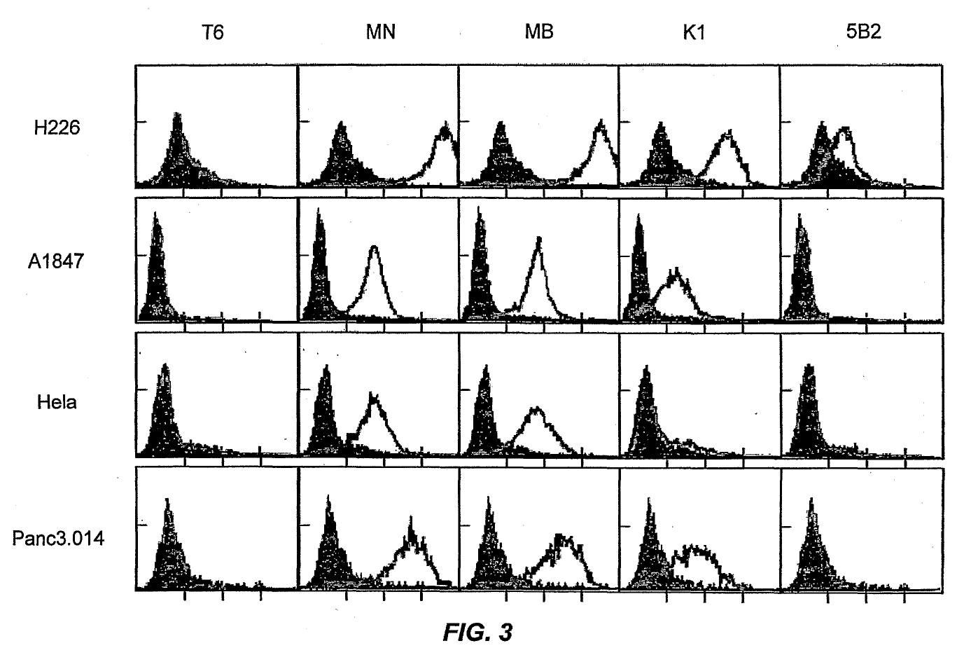

ELISA. To assess the reactivity of the MAbs in an ELISA format, plates coated with 2 μg/ml of mesothelin-Fc were exposed to increasing concentrations of each of the MAbs. FIG. 3A shows representative data from triplicate experiments. MAb MN showed the strongest signal followed by MAb MB. MAbs K1 and 5B2 showed a very weak signal. In a control ELISA using plates coated with CD25-Fc, none of the antibodies showed a signal. Thus, MAb MN appears to be the best choice for detection of mesothelin in this ELISA format. In another ELISA format plates were coated with mesothelin produced in E. coli and the reactivity of the antibodies compared. FIG. 3B shows representative data from triplicate experiments. MN showed a very strong signal followed by 5B2, which showed a weak signal, and K1 and MB showed a very weak signal.

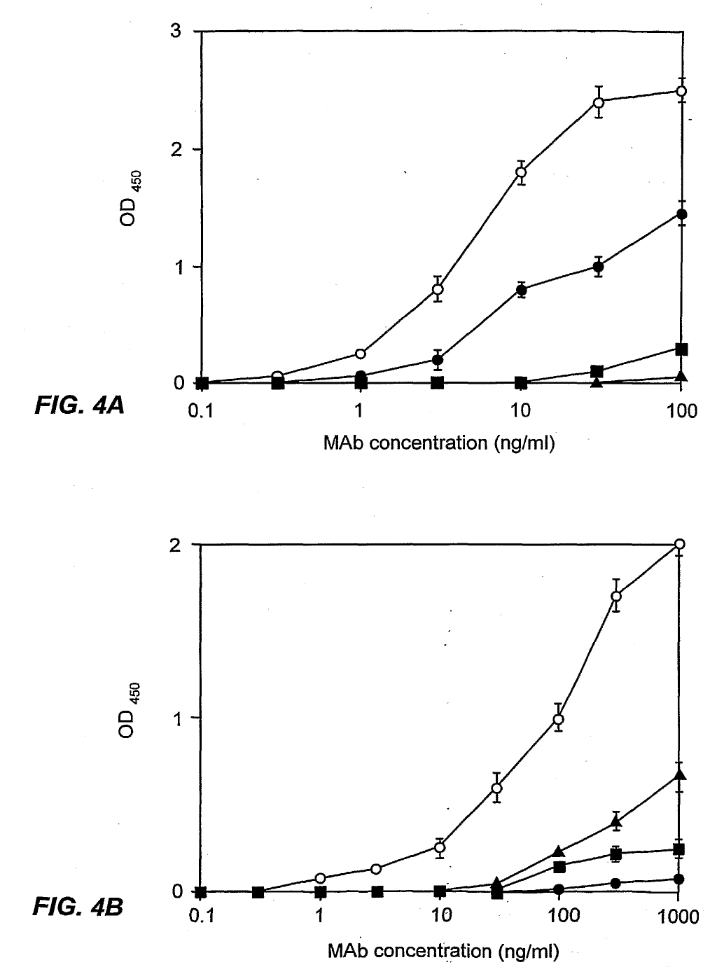

Western Blot. The reactivity of the anti-mesothelin MAbs to SDS-denatured antigen was tested in a Western blot analysis in which various amounts of mesothelin-Fc were loaded on a gel and blotted with each antibody. The data in FIG. 4 shows that MAbs MN and MB are 10 times more sensitive in detecting mesothelin on the Western blot than MAbs K1 and 5B2. For specificity controls, we showed that none of the antibodies reacted with the CD25-Fc control protein. In addition, monoclonal anti-CD30 antibodies did not react with mesothelin-Fc or CD25-Fc protein. This result suggests that these MAbs probably recognize a linear epitope on mesothelin.

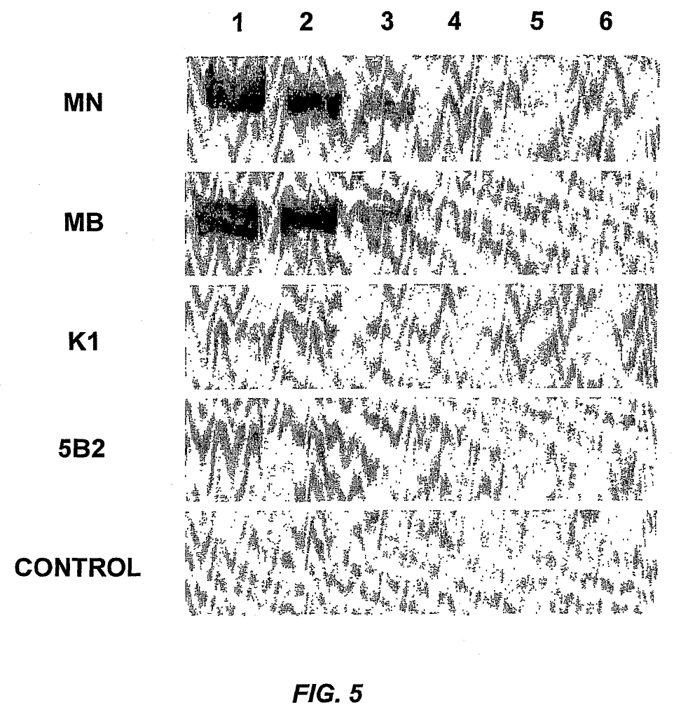

Immunohistochemistry. The patterns seen using peroxidase immunohistochemistry with different anti-mesothelin antibodies in the same area of the same case of mesothelioma are shown in FIG. 5. In panel A, sections treated with standard antigen retrieval show very poor reaction with a case of mesothelioma using MAb K1, whereas panel A′ shows the result following antigen retrieval using 3M urea. The levels of reactivities detected under these conditions are comparable to those seen in the detection of mesothelin using MAb K1 in frozen sections. Thus, MAb K1 can detect mesothelin expression, but only in frozen sections or paraffin sections treated using urea antigen retrieval, and is much less effective using standard antigen retrieval. On the other hand, MAb 5B2, a commercial antibody from Novocastra, shows good reactivity after standard antigen retrieval (panel B), a level of intensity similar to MAb MN shown in panel D. MAb MB, however, shows superior reactivity with standard antigen retrieval, as shown in panel C. This pattern was observed in three separate cases of mesothelioma examined using these reagents. This suggests that MAb MB is a superior reagent for use with standard immunohistochemical procedures in archival paraffin-embedded tissues.

TABLE 1
Characterization of anti-mesothelin MAbs

| | | Affin- | | | West- | | |
|---|---|---|---|---|---|---|---|
| | Iso- | ity^{†} | | ELISA^{§} | ern^{∥} | | Epi- |
| MAbs | type* | (nM) | FACS^{‡} | (ng/ml) | (ng) | IHC** | tope^{††} |
| MN | IgG2a | 1.0 | 3.5 | 1.1 | 2 | + | 1 |
| MB | IgG2a | 0.6 | 3.3 | 1.9 | 2 | + | 2 |
| K1 | IgG1 | 12 | 2.5 | 1000 | 25 | + | 1 |
| 5B2 | IgG1 | NA^{‡‡} | 1.3 | 1000 | 25 | + | NA |

*All MAbs contained a κ light chain.
^{†}Affinity to mesothelin-Fc in solution determined by Biacore.
^{‡}Reactivity to H226 cells in FACS (log geometric mean of fluorescence intensity). Each MAb (1 μg/ml) was incubated with H226 (mesothelin positive) cells and the bound MAb was detected by PE-labeled anti-mouse IgG. FACS histograms are shown in FIG. 2. The values are geometric means of FACS signals. All the anti-mesothelin MAbs reacted to H226 cells.
^{§}Reactivity to ELISA. ELISA plates were coated with mesothelin-Fc. After incubation with each MAbs, the bound MAb was detected by HRP-labeled anti-mouse IgG. The values are the amounts of MAbs that showed OD₄₅₀ = 0.5.
^{∥}Reactivity to SDS-denatured mesothelin-Fc in Western blot (FIG. 4). The values are the minimum amount of mesothelin-Fc from which each MAb can be detected with.
**Immunohistochemistry. The results of K1 and 5B2 were cited from the previous reports (Chang, K. et al., Int J Cancer 50: 373-81 (1992); Hassan, R. et al., Clin Cancer Res 10: 3937-42 (2004); Ordonez, N. G. Mol Pathol 16: 192-7 (2003)).
^{††}Topological group of epitopes identified based on the mutual competition of the MAbs (Nagata, S. et al., J Immunol Methods 292: 141-55 (2004)).
^{‡‡}Not applicable because 5B2 does not bind well to mesothelin-Fc.

### Example 3

The newly established MAbs react strongly and specifically to two different epitopes on the native form of the mesothelin as well as with denatured mesothelin. One of these epitopes was recognized by the K1 antibody previously made in normal mice. MAbs MN, K1 and immunotoxin SS1P, in which the Fv was obtained from an antibody phage library are in the same epitope group. MAb Kᵢ was generated by immunization with the human ovarian cancer cell line OVCAR-3 (Chang, K. et al., Am J Surg Pathol 16:259-68 (1992), Chang, K. et al., Int J Cancer 50:373-81 (1992)). On the other hand, the Fv of SS1P was cloned from the splenic mRNA of mice using antibody phase display. The mice were immunized with an expression vector coding for mesothelin. Fifteen MAbs of the 17 newly established MAbs reacted with this epitope. Because such antibodies are frequently generated by three different methods of immunization, this must be a dominant epitope (Chang, K. et al., Am J Surg Pathol 16:259-68 (1992); Kreitman, R. J. et al., N Engl J Med 345:241-7 (2001)). MAb MN reacts with mesothelin-Fc protein and with mesothelin made in E. coli, showing this epitope is present on recombinant mesothelin made in mammalian cells and in bacteria (FIG. 3). The epitope recognized by MAb MB does not react with mesothelin made in bacteria (FIG. 3B). MAb MB works very well for immunohistochemistry using standard antigen retrieval and should therefore be useful for such studies. On the other hand, antibody 5B2, a previously available antibody, mainly reacts with the bacterial form of mesothelin, but not with native mesothelin made in human cells.

The affinities of the new anti-mesothelin MAbs were measured by Biacore and found to be high affinity antibodies, with Kds of 1.0 and 0.6 nM. These antibodies showed good performance when used for FACS analysis, ELISA, Western blot and immunohistochemistry (Table 1). Thus, these MAbs are useful for detecting the mesothelin protein using all types of immunological assays. These new anti-mesothelin MAbs could be useful for detection of mesothelin as well as for antibody-based therapies.

In conclusion, we have established new anti-mesothelin MAbs that have better performance characteristics for immunohistochemistry and immunological assays than previously available antibodies.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. An isolated antibody comprising a variable heavy (V_{H}) chain and a variable light (V_{L}) chain, which V_{H} and V_{L} chains have 90% or greater identity to SEQ ID NOS:1 and 3, respectively, or to the V_{H} and V_{L} chains of antibody MB, ATCC Patent Deposit Designation PTA-6709, respectively, and which specifically binds mesothelin.

2. An antibody of claim 1, wherein said identity to SEQ ID NOS:1 and 3 or to the V_{H} and V_{L} chains of antibody MB, ATCC Patent Deposit Designation PTA-6709, respectively, is 95% or greater.

3. An antibody of claim 1, wherein said V_{H} and said V_{L} chains each have complementarity determining regions (CDRs) 1, 2, and 3, wherein
(a) CDRs 1, 2, and 3 of said V_{H} chain have the sequences shown in FIG. 1 with respect to SEQ ID NO:1 and which CDRs 1, 2, and 3 of said V_{L} chain have the sequences shown in FIG. 1, with respect to SEQ ID NO:3, or
(b) CDRs 1, 2, and 3 of said V_{H} chain have the sequences of the corresponding CDRs of antibody MB, ATCC Patent Deposit Designation PTA-6709, and which CDRs 1, 2, and 3 of said V_{L} chain have the sequences of the corresponding CDRs of antibody MB, ATCC Patent Deposit Designation PTA-6709.

4. An antibody of claim 1, which antibody comprises (a) SEQ ID NOS:1 and 3 joined by a peptide linker or, (b) the V_{H} and V_{L} chains of antibody MB, ATCC Patent Deposit Designation PTA-6709, joined by a peptide linker.

5. An antibody of claim 1, wherein said V_{H} and said V_{L} chains are connected by a disulfide bond between a cysteine residue in each of said chains.

6. An antibody of claim 1, wherein said antibody is selected from the group consisting of an scFv, a dsFv, a diabody, a domain antibody, a Fab, a F(ab′)₂ or an intact immunoglobulin.

7. An antibody of claim 1, wherein said V_{H} and said V_{L} chains each have complementarity determining regions (“CDRs”) 1, 2, and 3, wherein
CDRs 1, 2, and 3 of said V_{H} chain and CDRs 1, 2, and 3 of said V_{L} chain have the sequences shown in FIG. 1 for antibody MN or the sequence of the corresponding CDRs of antibody MB, ATCC Patent Deposit Designation PTA-6709, respectively, except that
(a) one or more CDRs have a mutation of a residue encoded by a codon with a nucleotide falling within (i) a tetranucleotide motif A/G-G-C/T-A/T or (ii) AGY, where Y can be a C or a T, or
(b) one or more CDRs have a mutation of a residue that is not encoded by a codon with a nucleotide falling within (i) a tetranucleotide motif A/G-G-C/T-A/T or (ii) AGY, where Y can be a C or a T, or
(c) one or more CDRs have a mutation of a residue that is encoded by a codon with a nucleotide falling within (i) a tetranucleotide motif A/G-G-C/T-A/T or (ii) AGY, where Y can be a C or a T, and one or more CDRs have a mutation of a residue that is not encoded by a codon with a nucleotide falling within (i) a tetranucleotide motif A/G-G-C/T-A/T or (ii) AGY, where Y can be a C or a T.

8. A chimeric molecule comprising (a) an isolated antibody which specifically binds mesothelin, which antibody comprises a variable heavy (V_{H}) chain and a variable light (V_{L}) chain, which V_{H} and V_{L} chains have 90% or greater identity to (i) SEQ ID NOS:1 and 3, respectively, or (ii) to the V_{H} and V_{L} chains of antibody MB, ATCC Patent Deposit Designation PTA-6709, respectively, and (b) a therapeutic moiety or a detectable label.

9. A chimeric molecule of claim 8, wherein the V_{H} and V_{L} chains have 95% or greater identity to (i) SEQ ID NOS:1 and 3, respectively, or (ii) to the V_{H} and V_{L} chains of antibody MB, ATCC Patent Deposit Designation PTA-6709, respectively.

10. A chimeric molecule of claim 8, wherein the V_{H} and V_{L} chains have the sequence of (i) SEQ ID NOS:1 and 3, respectively, or (ii) the V_{H} and V_{L} chains of antibody MB, ATCC Patent Deposit Designation PTA-6709, respectively.

11. A chimeric molecule of claim 8, wherein the therapeutic moiety is selected from the group consisting of a cytotoxin, a drug, a radioisotope, or a liposome loaded with a drug or a cytotoxin.

12. A chimeric molecule of claim 11, wherein the therapeutic moiety is a cytotoxin is selected from the group consisting of ricin A, abrin, ribotoxin, ribonuclease, saporin, calicheamycin, diphtheria toxin, a Pseudomonas exotoxin (“PE”), and botulinum toxins A through F.

13. A chimeric molecule of claim 8, wherein said V_{H} and V_{L} chains each have complementarity determining regions (“CDRs”) 1, 2, and 3, wherein
CDRs 1, 2, and 3 of said V_{H} chain and CDRs 1, 2, and 3 of said V_{L} chain have the sequences shown in FIG. 1 for antibody MN or the CDRs of antibody MB, ATCC Patent Deposit Designation PTA-6709, respectively, except that
(i) one or more CDRs have a mutation of a residue encoded by a codon with a nucleotide falling within (A) a tetranucleotide motif A/G-G-C/T-A/T or (B) AGY, where Y can be a C or a T, or
(ii) one or more CDRs have a mutation of a residue that is not encoded by a codon with a nucleotide falling within (A) a tetranucleotide motif A/G-G-C/T-A/T or (B) AGY, where Y can be a C or a T, or
(iii) one or more CDRs have a mutation of a residue that is encoded by a codon with a nucleotide falling within (A) a tetranucleotide motif A/G-G-C/T-A/T or (B) AGY, where Y can be a C or a T, and one or more CDRs have a mutation of a residue that is not encoded by a codon with a nucleotide falling within (C) a tetranucleotide motif A/G-G-C/T-A/T or (D) AGY, where Y can be a C or a T.

14. A composition comprising a chimeric molecule of claim 8 and a pharmaceutically acceptable carrier.

15. A composition of claim 14, wherein the therapeutic moiety is selected from the group consisting of a cytotoxin, a drug, a radioisotope, or a liposome loaded with a drug or a cytotoxin.

16. An isolated nucleic acid encoding an antibody comprising a variable heavy (V_{H}) chain and a variable light (V_{L}) chain, which V_{H} and V_{L} chains have 90% or greater identity to SEQ ID NOS:1 and 3, respectively, or to the V_{H} and V_{L} chains, respectively, of antibody MB, ATCC Patent Deposit Designation PTA-6709, respectively, and which specifically binds mesothelin.

17. A nucleic acid of claim 16, wherein said identity to SEQ ID NOS:1 and 3, respectively, or to the V_{H} and V_{L} chains, respectively, of antibody MB, ATCC Patent Deposit Designation PTA-6709, respectively, is 95% or greater.

18. A nucleic acid of claim 16, wherein the V_{H} and V_{L} chains have the sequence of (i) SEQ ID NOS:1 and 3, respectively, or (ii) the V_{H} and V_{L} chains, respectively, of antibody MB, ATCC Patent Deposit Designation PTA-6709.

19. A nucleic acid of claim 16, wherein said V_{H} and said V_{L} chains each have complementarity determining regions (CDRs) 1, 2, and 3, wherein
(a) CDRs 1, 2, and 3 of said V_{H} chain have the sequences shown in FIG. 1 with respect to SEQ ID NO:1 and which CDRs 1, 2, and 3 of said V_{L} chain have the sequences shown in FIG. 1, with respect to SEQ ID NO:3, or
(b) CDRs 1, 2, and 3 of said V_{H} chain have the sequences of CDRs 1, 2, and 3, respectively, of the V_{H} chain of antibody MB, ATCC Patent Deposit Designation PTA-6709 and which CDRs 1, 2, and 3 of said V_{L} chain have the sequences of CDRs 1, 2, and 3, respectively, of the V_{L} chain of antibody MB, ATCC Patent Deposit Designation PTA-6709.

20. A nucleic acid of claim 16, wherein said V_{H} and said V_{L} chains each have complementarity determining regions (CDRs) 1, 2, and 3, wherein CDRs 1, 2, and 3 of said V_{H} chain and CDRs 1, 2, and 3 of said V_{L} chain have the sequences shown in FIG. 1 for antibody MN, or of antibody MB, ATCC Patent Deposit Designation PTA-6709, respectively, except that
(i) one or more CDRs have a mutation of a residue encoded by a codon with a nucleotide falling within (A) a tetranucleotide motif A/G-G-C/T-A/T or (B) AGY, where Y can be a C or a T, or
(ii) one or more CDRs have a mutation of a residue that is not encoded by a codon with a nucleotide falling within (A) a tetranucleotide motif A/G-G-C/T-A/T or (B) AGY, where Y can be a C or a T, or
(iii) one or more CDRs have a mutation of a residue that is encoded by a codon with a nucleotide falling within (A) a tetranucleotide motif A/G-G-C/T-A/T or (B) AGY, where Y can be a C or a T, and one or more CDRs have a mutation of a residue that is not encoded by a codon with a nucleotide falling within (C) a tetranucleotide motif A/G-G-C/T-A/T or (D) AGY, where Y can be a C or a T.

21. A nucleic acid of claim 16, wherein said antibody encoded by said nucleic acid is selected from the group consisting of an scFv, a dsFv, a Fab, a F(ab′)₂, a diabody, a domain antibody, or an intact immunoglobulin.

22. A nucleic acid of claim 16, wherein said nucleic acid further encodes a therapeutic moiety or a detectable label.

23. A nucleic acid of claim 22, wherein said therapeutic moiety is a drug or a cytotoxin.

24. A nucleic acid of claim 23, further wherein said cytotoxin is selected from the group consisting of ricin A, abrin, ribotoxin, ribonuclease, saporin, calicheamycin, diphtheria toxin, a Pseudomonas exotoxin (“PE”), and botulinum toxins A through F.

25. An expression vector comprising a nucleic acid of claim 16 operably linked to a promoter.

26. An expression vector of claim 25, wherein said nucleic acid further encodes a therapeutic moiety or a detectable label.

27. A method of inhibiting growth of a cell expressing mesothelin by contacting said cell with a chimeric molecule comprising (a) an antibody that binds to mesothelin, which antibody has variable heavy (V_{H}) and variable light (V_{L}), which V_{H} and V_{L} chains have 90% or greater identity to SEQ ID NOS:1 and 3, respectively, or to the V_{H} and V_{L} chains of antibody MB, ATCC Patent Deposit Designation PTA-6709, respectively, and (b) a therapeutic moiety,
whereby contacting said cell with said therapeutic moiety inhibits growth of said cell.

28. A method of claim 27, wherein said identity to SEQ ID NOS:1 and 3 or to the V_{H} and V_{L} chains, respectively, of antibody MB, ATCC Patent Deposit Designation PTA-6709, is 95% or greater.

29. A method of claim 27, wherein said identity to SEQ ID NOS:1 and 3 or to the V_{H} and V_{L} chains, respectively, of antibody MB, ATCC Patent Deposit Designation PTA-6709, is 95% or greater.

30. A method of claim 27, wherein said V_{H} and V_{L} chains have the sequence of SEQ ID NOS:1 and 3, respectively, or of the V_{H} and V_{L} chains, respectively, of antibody MB, ATCC Patent Deposit Designation PTA-6709.

31. A method of claim 27, wherein said V_{H} and said V_{L} chains each have complementarity determining regions (CDRs) 1, 2, and 3, wherein
(a) CDRs 1, 2, and 3 of said V_{H} chain have the sequences shown in FIG. 1 with respect to SEQ ID NO:1 and which CDRs 1, 2, and 3 of said V_{L} chain have the sequences shown in FIG. 1, with respect to SEQ ID NO:3, or
(b) CDRs 1, 2, and 3 of said V_{H} chain have the sequences of the CDRs of the V_{H} chain of antibody MB, ATCC Patent Deposit Designation PTA-6709, and CDRs 1, 2, and 3 of said V_{L} chain have the sequences of the CDRs of the V_{L} chain of antibody MB, ATCC Patent Deposit Designation PTA-6709.

32. A method of claim 31, wherein said V_{H} and said V_{L} chains each have complementarity determining regions (CDRs) 1, 2, and 3, wherein CDRs 1, 2, and 3 of said V_{H} chain and CDRs 1, 2, and 3 of said V_{L} chain have the sequences shown in FIG. 1 for antibody MN, or of the CDRs of the respective chain of antibody MB, ATCC Patent Deposit Designation PTA-6709, respectively, except that
(i) one or more CDRs have a mutation of a residue encoded by a codon with a nucleotide falling within (A) a tetranucleotide motif A/G-G-C/T-A/T or (B) AGY, where Y can be a C or a T, or
(ii) one or more CDRs have a mutation of a residue that is not encoded by a codon with a nucleotide falling within (A) a tetranucleotide motif A/G-G-C/T-A/T or (B) AGY, where Y can be a C or a T, or
(iii) one or more CDRs have a mutation of a residue that is encoded by a codon with a nucleotide falling within (A) a tetranucleotide motif A/G-G-C/T-A/T or (B) AGY, where Y can be a C or a T, and one or more CDRs have a mutation of a residue that is not encoded by a codon with a nucleotide falling within (C) a tetranucleotide motif A/G-G-C/T-A/T or (D) AGY, where Y can be a C or a T.

33. A method of claim 27, wherein said antibody is selected from the group consisting of an scFv, a dsFv, a Fab, a F(ab′)₂, a diabody, a domain antibody, or an intact immunoglobulin.

34. A method of claim 27, wherein said therapeutic moiety is selected from the group consisting of a cytotoxin, a drug, a radioisotope, or a liposome loaded with a drug or a cytotoxin.

35. A method of claim 34, wherein the cytotoxin is selected from the group consisting of ricin A, abrin, ribotoxin, ribonuclease, saporin, calicheamycin, diphtheria toxin, a Pseudomonas exotoxin (“PE”), and botulinum toxins A through F.

36. A method for detecting the presence of a cell expressing mesothelin in a biological sample, said method comprising:
(a) contacting cells of said biological sample with a chimeric molecule comprising (i) an antibody that specifically binds to mesothelin, said antibody having a variable heavy (V_{H}) chain and a variable light (V_{L}) chain, which V_{H} and V_{L} chains have 90% or greater identity to SEQ ID NOS:1 and 3, respectively, or to the VH chain and the VL chain, respectively, of antibody MB, ATCC Patent Deposit Designation PTA-6709 SEQ ID NOS:1 and 3, and (ii) a detectable label; and,
(b) detecting the presence or absence of said label, wherein detecting the presence of said label indicates the presence of a mesothelin-expressing cell in said sample.

37. A method of claim 36, wherein said identity to SEQ ID NOS:1 and 3 or to the V_{H} chain and of the V_{L} chain of antibody MB, ATCC Patent Deposit Designation PTA-6709, respectively, is 95% or greater.

38. A method of claim 36, wherein said VH chain and said VL chain have the sequence of (i) SEQ ID NOS:1 and 3, respectively, or (ii) or of the V_{H} chain and of the V_{L} chain, respectively, of antibody MB, ATCC Patent Deposit Designation PTA-6709.

39. A method of claim 36, wherein said V_{H} and said V_{L} chains each have complementarity determining regions (CDRs) 1, 2, and 3, wherein
(a) CDRs 1, 2, and 3 of said V_{H} chain have the sequences shown in FIG. 1 with respect to SEQ ID NO:1 and which CDRs 1, 2, and 3 of said V_{L} chain have the sequences shown in FIG. 1, with respect to SEQ ID NO:3, or
(b) CDRs 1, 2, and 3 of said V_{H} chain have the sequences of the corresponding CDRs of the V_{H} chain of antibody MB, ATCC Patent Deposit Designation PTA-6709 and which CDRs 1, 2, and 3 of said V_{L} chain have the sequences of the corresponding CDRs of antibody MB, ATCC Patent Deposit Designation PTA-6709.

40. A method of claim 36, wherein said V_{H} and said V_{L} chains each have complementarity determining regions (CDRs) 1, 2, and 3, wherein CDRs 1, 2, and 3 of said V_{H} chain and CDRs 1, 2, and 3 of said V_{L} chain have the sequences shown in FIG. 1 for antibody MN, respectively, or the sequences of the corresponding CDRs of antibody MB, ATCC Patent Deposit Designation PTA-6709, respectively, except that
(i) one or more CDRs have a mutation of a residue encoded by a codon with a nucleotide falling within (A) a tetranucleotide motif A/G-G-C/T-A/T or (B) AGY, where Y can be a C or a T, or
(ii) one or more CDRs have a mutation of a residue that is not encoded by a codon with a nucleotide falling within (A) a tetranucleotide motif A/G-G-C/T-A/T or (B) AGY, where Y can be a C or a T, or
(iii) one or more CDRs have a mutation of a residue that is encoded by a codon with a nucleotide falling within (A) a tetranucleotide motif A/G-G-C/T-A/T or (B) AGY, where Y can be a C or a T, and one or more CDRs have a mutation of a residue that is not encoded by a codon with a nucleotide falling within (C) a tetranucleotide motif A/G-G-C/T-A/T or (D) AGY, where Y can be a C or a T.

41. A method of claim 36, wherein said antibody is selected from the group consisting of an scFv, a dsFv, a Fab, a F(ab′)₂, a diabody, a domain antibody, or an intact immunoglobulin.

42. A kit for detecting the presence of a mesothelin-expressing cell in a biological sample, said kit comprising:
(a) a container, and
(b) a chimeric molecule comprising (i) an antibody that specifically binds to mesothelin, said antibody having a variable heavy (V_{H}) chain and a variable light (V_{L}) chain, which V_{H} and V_{L} chains have 90% or greater identity to SEQ ID NOS:1 and 3, respectively or to the sequences of the V_{H} and the V_{L} chains, respectively, of antibody MB, ATCC Patent Deposit Designation PTA-6709.

43. A kit of claim 42, wherein said identity to SEQ ID NOS:1 and 3 or to the sequences of the V_{H} and the V_{L} chains, respectively, of antibody MB, ATCC Patent Deposit Designation PTA-6709, is 95% or greater.

44. A kit of claim 42, wherein said antibody V_{H} and V_{L} chains have the sequence of SEQ ID NOS:1 and 3, respectively or the sequences of the V_{H} and the V_{L} chains, respectively, of antibody MB, ATCC Patent Deposit Designation PTA-6709.

45. A kit of claim 42, wherein said V_{H} chain and said V_{L} chain each have complementarity determining regions (CDRs) 1, 2, and 3, wherein
(a) CDRs 1, 2, and 3 of said V_{H} chain have the sequences shown in FIG. 1 with respect to SEQ ID NO:1 and which CDRs 1, 2, and 3 of said V_{L} chain have the sequences shown in FIG. 1, with respect to SEQ ID NO:3, or
(b) CDRs 1, 2, and 3 of said V_{H} chain and which CDRs 1, 2, and 3 of said V_{L} chain have the sequences of the corresponding chain of antibody MB, ATCC Patent Deposit Designation PTA-6709.

46. A kit of claim 42, wherein said V_{H} and said V_{L} chains each have complementarity determining regions (CDRs) 1, 2, and 3, wherein CDRs 1, 2, and 3 of said V_{H} chain and CDRs 1, 2, and 3 of said V_{L} chain have the sequences shown in FIG. 1 for antibody MN or CDRs 1, 2, and 3 of the corresponding chain of antibody MB, ATCC Patent Deposit Designation PTA-6709, for antibody MB, respectively, except that
(i) one or more CDRs have a mutation of a residue encoded by a codon with a nucleotide falling within (A) a tetranucleotide motif A/G-G-C/T-A/T or (B) AGY, where Y can be a C or a T, or
(ii) one or more CDRs have a mutation of a residue that is not encoded by a codon with a nucleotide falling within (A) a tetranucleotide motif A/G-G-C/T-A/T or (B) AGY, where Y can be a C or a T, or
(iii) one or more CDRs have a mutation of a residue that is encoded by a codon with a nucleotide falling within (A) a tetranucleotide motif A/G-G-C/T-A/T or (B) AGY, where Y can be a C or a T, and one or more CDRs have a mutation of a residue that is not encoded by a codon with a nucleotide falling within (C) a tetranucleotide motif A/G-G-C/T-A/T or (D) AGY, where Y can be a C or a T.

47. A kit of claim 42, further comprising a detectable label.
